(19) United States
(12) Patent Application Publication
(10) Pub. No.: US 2021/0228311 A1
(43) Pub. Date: Jul. 29, 2021

(54) ATTACHMENT APPARATUS FOR A BODY MOUNTABLE MEDICAL DEVICE

(71) Applicant: XACT ROBOTICS LTD., Caesarea (IL)

(72) Inventors: Ben GALILI, Atlit (IL); Eli LEVIN, Haifa (IL); Yoav TIKOCHINSKY, Tel Aviv (IL); Idan ZILZER, Tel Aviv (IL)

(21) Appl. No.: 17/059,443

(22) PCT Filed: Jun. 6, 2019
(86) PCT No.: PCT/IL2019/050651

§ 371 Date: Nov. 29, 2020

Related U.S. Application Data
(60) Provisional application No. 62/763,226, filed on Jun. 7, 2018.

(51) Int. Cl. A61B 90/50; A61B 90/11

(52) U.S. Cl. CPC A61B 90/50; A61B 2560/04; A61B 90/11

(57) ABSTRACT

There is provided herein, an attachment apparatus for attaching a medical device to a body of a subject, the apparatus including: an attachment frame configured for positioning on the body of the subject and for receiving the medical device thereon; one or more extending members coupleable to the attachment frame and extendable essentially sideways therefrom; one or more straps; and one or more strap connectors configured to couple the one or more straps to at least one of the attachment frame and the one or more extending members. There is further provided herein a method for attaching a medical device to a body of a subject utilizing the attachment apparatus.

### TECHNICAL FIELD

The present disclosure relates to the field of medical procedures, specifically to devices and methods for attaching a medical device to a body of a subject, and more specifically, to an attachment frame for a body mountable medical device.

### BACKGROUND

Many routine treatments employed in modern clinical practice involve percutaneous insertion of medical tools, such as needles and catheters, for biopsy, drug delivery and other diagnostic and therapeutic procedures. The aim of an insertion procedure is to place the tip of an appropriate medical tool safely and accurately in a target region, which could be a lesion, tumor, organ or vessel. Examples of treatments requiring insertion of such medical tools include vaccinations, blood/fluid sampling, regional anesthesia, tissue biopsy, catheter insertion, cryogenic ablation, electrolytic ablation, brachytherapy, neurosurgery, deep brain stimulation and various minimally invasive surgeries.

Guidance and steering of needles in soft tissue is a complicated task that requires good three-dimensional coordination, knowledge of the patient anatomy and a high level of experience. Therefore, image-guided automated (e.g., robotic) systems have been proposed for performing these functions. Among such systems are those described in U.S. Pat. No. 7,008,373 to Stoianovici, for “System and method for robot targeting under fluoroscopy”, and in U.S. Pat. No. 8,348,861 to Glozman et al, for “Controlled Steering of a Flexible Needle”.

In recent years, body mounted automated medical devices and methods for planning their trajectories have been introduced. Some of these devices are guiding devices that assist in choosing the insertion point and/or in aligning the needle with the insertion point and with the target and the physician then inserts the needle manually, and some are devices that also insert the needle towards the target, as disclosed, for example, in U.S. Application Publication No. 2006/0229641 to Gupta et al, for “Guidance and Insertion System”, U.S Application Publication No. 2009/0112119 to Kim, for “Rotating Biopsy Device and Biopsy Robot”, U.S. Pat. No. 9,326,825 to Cleary et al, for “Patient Mounted MRI and CT Compatible Robot for Needle Guidance in Interventional Procedures” and U.S. Patent Application Publication No. 2016/0249990 to Glozman et al, for “Needle Steering by Shaft Manipulation”.

Attaching the medical device to the patient's body is advantageous since it compensates for patient motion during the procedure as the device moves together with the patient. This minimizes tissue damage and patient discomfort which may be caused by relative movement between the device and the patient.

Gupta et al discloses a guidance and insertion device that is attached to the patient's body using tabs that tape the device's support base to the tissue surface or that include slots for receiving straps that secure the support base onto the patient. Kim discloses a portable biopsy robot that is attached to the patient's skin using a strong adhesive located on the bottom of the robot. Cleary et al discloses a patient mountable robot that is attached to the patient's body using a four-adjustable-leg mechanism, each of the legs having an adhesive pad that directly contacts and sticks to the patient's body. Glozman et al discloses a robotic needle manipulator that is attached to the patient's body using belts or adhesives.

Direct attachment to the patient's skin using adhesives prevents a much-desired flexibility in positioning the automated device on the patient's body, and in re-positioning the device if necessary. Further, the use of adhesives does not allow placement of a sterile drape between the device and the patient's body in case the device is reusable and non-sterile, and may cause discomfort and skin irritation to the patient.

Thus, there is a need for improved devices and methods for attaching a medical device to a body of a subject.

The disclosures of each of the publications mentioned in this section and in other sections of the specification, are hereby incorporated by reference, each in its entirety.

### SUMMARY

The present disclosure describes an apparatus for attaching a medical device (e.g., an insertion device) to the subject's body.

When a medical device is attached to the subject's body using belts or straps, it is important to moderate the strapping force, as it effects the loads on the subject, which may cause displacement of internal soft tissues as well as discomfort to the subject. As there is friction between the straps and the subject's skin, some of the total strapping force is used to overcome the force created by friction. Therefore, the attachment solution should strive to minimize the total strapping loads, by maximizing the mounting area and minimizing the strapping force losses.

Maximizing the mounting area may be achieved by using an intermediary element, such as a mounting base (also referred to as “attachment frame” or “mediator plate”), as disclosed, for example, in co-owned U.S. patent application Ser. No. 16/092,786, to Arnold et al. Thus, an attachment apparatus of the present disclosure may comprise a mounting base configured to be secured to the subject's body, e.g., using straps (also referred to as “belts”), and to receive the medical device thereon. The medical device may be secured to the mounting base using securing elements, such as latches.

Typically, when straps 13 are used to secure the mounting base 12 to the patient's body, the straps 13 surround the patient's body 5 and maintain direct contact with the patient's skin along substantially the entire circumference of the body, as shown in FIG. 1, or along the distance between the mounting base 12 and a back base positioned between the patient's body and the patient bed 17, as disclosed, for example, in abovementioned U.S. patent application Ser. No. 16/092,786. The force “F” applied by the straps 13 on the mounting base 12 can be viewed as a summation vector of the force vertical to the mounting base, F_{y}, and the force parallel to the mounting base, Fₓ. Since the force parallel to the mounting base 12 has no added value to the attachment of the mounting base 12 to the body 5, the equivalent force vector applied by the straps 13 on the mounting base 12 is maximized when the straps 13 are orthogonal to the mounting base 12.

In some embodiments, the medical device is an insertion device configured to insert a medical tool, such as a needle or an introducer, into the body of the patient, for the purpose of performing, for example, a biopsy or an ablation procedure, or for delivering a substance, such as medication, into the body. In such cases, the required insertion force is typically predetermined, and hence the required vertical force applied to the mounting base is also predetermined, as these forces must be substantially equal to prevent movement of the mounting base and the coupled insertion device (assuming no relative movement therebetween), while executing the insertion procedure. If the mounting base is attached to the subject's body such that only the vertical force is applied to the mounting base, the strapping force losses are minimized, and thus the total strapping force and the load/pressure sensed by the subject, are minimized.

In some embodiments, the attachment apparatus may comprise one or more extending members (also referred to as “cranes”) coupled to the attachment frame at one or both ends of the frame, such that at least one strap is coupled to the attachment frame via a crane. The extending members may be separate units removably coupleable to the attachment frame using dedicated connectors, e.g., latches, or they may be coupled to the attachment frame during assembly. Alternatively, the attachment frame and at least one of the one or more extending members may be integrally formed. In some embodiments, the length of the cranes may be adjustable, to enable coupling of the straps to the attachment frame such that they are substantially orthogonal to the frame, thus maximizing the equivalent force vector applied by the straps on the attachment frame, and, generally, the cranes add a level of flexibility to the task of positioning the attachment frame on the subject.

Further, since the straps are substantially orthogonal to the attachment frame when the cranes are utilized, the contact area between the straps and the subject's skin is minimized, and thus, the friction losses are minimized, resulting in the decrease of the total strapping force needed to achieve the mandatory minimal effective mounting force of the attachment frame.

There is provided herein, in accordance with some embodiments, an attachment apparatus for attaching a medical device to a body of a subject, the apparatus including: an attachment frame configured for positioning on the body of the subject and for receiving the medical device thereon; one or more extending members coupleable to the attachment frame and extendable essentially sideways therefrom; one or more straps; and one or more strap connectors configured to couple the one or more straps to at least one of the attachment frame and the one or more extending members.

According to some embodiments, the one or more extending members may be configured as elongated members.

According to some embodiments, the attachment frame may include at least one channel, and wherein at least one of the one or more extending members is reversibly deployable from within the at least one channel. The attachment frame may include at least one locking member configured to lock the position of the at least one of the one or more extending members within the at least one channel. The at least one locking member may be rotatable.

According to some embodiments, the at least one of the one or more extending members may be configured as a telescopic member.

According to some embodiments, at least one of the one or more strap connectors may be configured to enable pivoting of at least one of the one or more straps coupled thereto relative to the at least one of the attachment frame and the one or more extending members.

According to some embodiments, at least one of the one or more strap connectors may either include or be coupled to a stretching mechanism. According to some embodiments, the at least one of the one or more strap connectors may include a first portion configured to be coupled to the at least one of the attachment frame and the one or more extending members, and a second portion configured to be coupled to at least one of the one or more straps and moveable relative to the first portion along a longitudinal axis of the at least one of the one or more strap connectors, and wherein the stretching mechanism may include at least one elastic member coupled to both of the first and second portions. According to some embodiments, the at least one of the one or more strap connectors may further include an indicator configured to indicate a stretching degree of the stretching mechanism. The indicator may include an indication bar and at least one marking. The indicator may include a rotatable member.

According to some embodiments, at least one of the one or more strap connectors may include a locking mechanism configured to lock the position of at least one of the one or more straps relative to the at least one of the one or more strap connectors. The locking mechanism may include at least one of: a rotating element, a puncturing element and a ratchet mechanism.

According to some embodiments, the at least one of the attachment frame and the one or more extending members may include one or more couplers configured to engage with the one or more strap connectors. The one or more strap connectors may include ball connectors, and the one or more couplers may include socket connectors configured to receive the ball connectors. The attachment frame may include two frame couplers positioned at a first end of the attachment frame and configured to engage with the one or more strap connectors, and wherein two extending members are coupled to the attachment frame at a second end of the attachment frame, each of the two extending members may include an extending member coupler configured to engage with the one or more strap connectors.

According to some embodiments, the attachment apparatus may further include a harness configured for positioning between the body of the subject and a surface configured for the subject to lie thereon. The attachment apparatus may further include one or more harness connectors configured to couple the one or more straps to the harness. The one or more harness connectors may include an inner space adapted to receive therein an edge of the harness. The one or more harness connectors may include a gripper configured to hold the edge of the harness within the inner space.

According to some embodiments, the attachment frame may include one or more alignment elements configured to engage with corresponding alignment members of the medical device upon positioning the medical device on the attachment frame, to facilitate proper alignment between the medical device and the attachment frame. The one or more alignment elements of the attachment frame may include one or more grooves, and the one or more alignment members of the medical device may include protrusions receivable within the one or more grooves.

According to some embodiments, the attachment frame may include a first portion of a coupling mechanism configured to couple the medical device to the attachment frame. The first portion of the coupling mechanism may be configured to engage with a second portion of the coupling mechanism, the second portion of the coupling mechanism being part of the medical device. According to some embodiments, at least one of the first and second portions of the coupling mechanism may include one or more indicators configured to indicate at least proper engagement of the first and second portions of the coupling mechanism. The first portion of the coupling mechanism may include one or more notches, and the second portion of the coupling mechanism may include one or more latches. According to some embodiments, at least one of the first and second portions of the coupling mechanism may include a safety mechanism configured to prevent engagement between the first and second portions of the coupling mechanism if the medical device in not properly positioned on the attachment frame. The safety mechanism may include a stopper configured to be disabled upon engagement of one or more alignment elements of the attachment frame with one or more corresponding alignment members of the medical device.

According to some embodiments, the attachment frame may include at least one sensor configured to detect movement of the attachment frame relative to the body of the subject and to generate an alert if the relative movement exceeds a predefined threshold. According to some embodiments, the at least one sensor may include one or more of: a distance sensor, a proximity sensor and an accelerometer.

According to some embodiments, the attachment apparatus may further include a lifting member configured to controllably lift the medical device from the attachment frame in one or more of a parallel manner and an angular manner. The lifting member may include at least one rail and at least one fixator configured to fixate the position of the medical device along the at least one rail. The lifting member may be removably coupleable to the attachment frame. The lifting member may rigidly be coupled to, or an integral part of, the attachment frame.

According to some embodiments, the attachment apparatus may further include at least one pad coupleable to at least a portion of the bottom surface of the attachment frame.

According to some embodiments, the medical device may be an insertion device, which includes a medical tool configured for insertion into the body of the subject, and wherein the attachment apparatus may further include an aiming apparatus removably coupleable to the attachment frame, the aiming apparatus being configured to facilitate proper positioning of the attachment frame relative to an entry point for entry of the medical tool into the body of the subject. According to some embodiments, the aiming apparatus may include a base having an opening configured for positioning above the entry point. The aiming apparatus may further include at least one coupling member configured to couple the aiming apparatus to the attachment frame. According to some embodiments, the at least one coupling member may include at least one latch configured to engage with at least one notch of the attachment frame. The aiming apparatus may further include an orienting mechanism configured to facilitate proper orienting of the attachment frame to enable insertion of the medical tool into the body of the subject, at the entry point, at a predetermined entry angle. The orienting mechanism may include a rotating arm, an upper arch, and an orienting member configured to be coupled to the rotating arm at its lower portion and to the upper arch at its top portion. The orienting mechanism may include a first locking element configured to prevent rotation of the rotating arm about its axis. The orienting mechanism may include a second locking element configured to prevent pivoting of the orienting member about an axis of a hinge coupling the orienting member to the rotating arm.

There is further provided herein, according to some embodiments, a method for attaching a medical device to a body of a subject, the method includes: providing an attachment apparatus essentially as described herein (each option is a separate embodiment); positioning the attachment frame on the body of the subject; using the one or more strap connectors, coupling the one or more straps to the at least one of the attachment frame and the one or more extending members; and coupling the medical device to the attachment frame.

According to some embodiments, the method may further include a step of deploying at least one of the one or more extending members from within at least one channel of the attachment frame.

According to some embodiments, the method may further include a step of locking the position of the at least one of the one or more extending members within the at least one channel, using at least one locking member of the attachment frame.

According to some embodiments, the method may further include a step of coupling the one or more straps to a harness configured for positioning between the body of the subject and a surface configured for the subject to lie thereon, using one or more harness connectors.

According to some embodiments, the method may further include a step of coupling an aiming apparatus to the attachment frame.

According to some embodiments, the method may further include a step of adjusting at least one of the height and the angle of the medical device relative to the attachment frame, using a lifting member.

There is further provided herein, according to some embodiments, an attachment apparatus for attaching a medical device to a body of a subject, the apparatus includes: a back base configured for positioning between the body of the subject and a surface configured for the subject to lie thereon; an attachment frame configured for positioning on the body of the subject and for receiving the medical device thereon, the attachment frame includes at least one channel; one or more extending members, at least one of the one or more extending members being configured for reversible deployment from within the at least one channel; one or more straps configured to be connected to the back base and to at least one of the attachment frame and the one or more extending members; one or more first connectors configured to connect a first end of the one or more straps to the at least one of the attachment frame and the one or more extending members; and one or more second connectors configured to connect a second end of the one or more straps to the back base.

According to some embodiments, the attachment frame may include at least one locking member configured to lock the position of the at least one of the one or more extending members within the at least one channel.

According to some embodiments, at least one of the one or more first connectors is configured to enable pivoting of at least one of the one or more straps coupled thereto relative to the at least one of the attachment frame and the one or more extending members.

According to some embodiments, the at least one of the attachment frame and the one or more extending members may include one or more couplers configured to engage with the one or more first connectors. The one or more first connectors may include ball connectors, and the one or more couplers may include socket connectors configured to receive the ball connectors. The attachment frame may include two frame couplers positioned at a first end of the attachment frame and configured to engage with the one or more first connectors, and wherein two extending members are coupled to the attachment frame at a second end of the attachment frame, each of the two extending members may include an extending member coupler configured to engage with the one or more first connectors.

According to some embodiments, at least one of the one or more first connectors either includes or is coupled to a stretching mechanism.

According to some embodiments, at least one of the one or more second connectors either includes or is coupled to a stretching mechanism.

According to some embodiments, the attachment frame may include one or more alignment elements configured to engage with corresponding alignment members of the medical device upon positioning the medical device on the attachment frame, to facilitate proper alignment between the medical device and the attachment frame.

According to some embodiments, the attachment frame may include a first portion of a coupling mechanism configured to couple the medical device to the attachment frame by engaging with a second portion of the coupling mechanism, the second portion of the coupling mechanism being part of the medical device. The first portion of the coupling mechanism may include one or more notches, and the second portion of the coupling mechanism may include one or more latches. According to some embodiments, at least one of the first and second portions of the coupling mechanism may include one or more indicators configured to indicate at least proper engagement of the first and second portions of the coupling mechanism.

According to some embodiments, the attachment frame and the one or more extending members may be integrally formed.

According to some embodiments, the one or more extending members may be configured to be coupled to the attachment frame.

It is to be understood that although some examples used throughout this disclosure relate to attachment of insertion devices to the body of a subject, the disclosed attachment apparatus is not limited for use with insertion devices alone and may be used with any medical device that is intended for attachment to the subject's body. Further, it can be appreciated that “attachment” of the medical device to the subject's body is to be interpreted as including direct attachment to the body, as well as attachment to the body via an intermediary element, such as a mounting base and/or cushion, etc.

Also, it is to be understood that although some examples used throughout this disclosure relate to insertion of a needle into a subject's body, this is done for simplicity reasons alone, and the scope of this disclosure is not limited to attachment of devices for insertion of a needle alone to the patient's body, but may include attachment of devices for insertion of any tool intended to be inserted into a subject's body for diagnostic and/or therapeutic purposes, including a needle, port, introducer, catheter (e.g., ablation catheter), cannula, surgical tool, fluid delivery tool, or any other such insertable tool.

In addition, it is to be understood that the term “strap” used throughout this disclosure to describe means for securing a medical device to the patient's body, may include any element suitable to be wrapped around at least part of the patient's body, such as strings, wires, cables, etc.

Finally, the terms “user”, “physician” and “clinician” are used interchangeably throughout this disclosure and they may refer to any person taking part in the performed medical procedure.

### BRIEF DESCRIPTION OF THE DRAWINGS

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1 is a schematic cross-sectional view of a patient's body with a typical medical device/mounting base secured thereto using straps;

FIG. 2 is a schematic perspective view of an exemplary attachment apparatus comprising a mediator plate and cranes, according to some embodiments;

FIGS. 3A and 3B are schematic perspective views of an exemplary attachment apparatus comprising a mediator plate and deployable cranes in a closed state (FIG. 3A) and an open state (FIG. 3B), according to some embodiments;

FIGS. 4A and 4B are perspective top (FIG. 4A) and bottom (FIG. 4B) views of an exemplary attachment apparatus comprising an attachment frame and deployable cranes with a locking mechanism, according to some embodiments;

FIG. 4C is a cross-sectional view of a crane of FIGS. 4A and 4B, extending out of a channel of the attachment frame and being locked in its extended position, according to some embodiments;

FIG. 5A is a front view of the medical device and the attachment frame of FIGS. 4A and 4B, prior to coupling, according to some embodiments;

FIGS. 5B and 5C are perspective front (FIG. 5B) and bottom (FIG. 5C) views of the medical device and the attachment frame of FIG. 5A after the medical device has been placed on the attachment frame, but before being secured thereto, according to some embodiments;

FIGS. 6A and 6B are perspective views of a medical device and an attachment frame securely coupled to one another, according to some embodiments;

FIGS. 7A-7C show examples of an aiming apparatus coupled to an attachment frame, according to some embodiments;

FIGS. 8A and 8B show an exemplary aiming apparatus comprising an orienting mechanism, according to some embodiments;

FIG. 9A shows an exemplary ball buckle used to connect a strap to a crane and/or to an attachment frame, according to some embodiments;

FIGS. 9B and 9C demonstrate exemplary rotation angles of an exemplary ball buckle used to connect a strap to a crane and/or to an attachment frame, according to some embodiments;

FIG. 10 shows an exemplary buckle used to connect a strap to a crane and/or to an attachment frame, the buckle comprising two parts which are moveable relative to each other, according to some embodiments;

FIGS. 11A-11C show an exemplary buckle used to connect a strap to a crane and/or to an attachment frame, the buckle comprising two parts which are moveable relative to each other, and having a stretching level indicator, according to some embodiments;

FIGS. 12A and 12B show another exemplary buckle comprising two parts which are moveable relative to each other, and having a stretching level indicator, according to some embodiments;

FIG. 13 is a side view of an exemplary ball buckle in which the ball connector is connected to the buckle using an arm, according to some embodiments;

FIGS. 14A and 14B show an exemplary buckle having elastic properties, according to some embodiments;

FIGS. 15A and 15B show an exemplary buckle comprising a locking handle for the strap, according to some embodiments;

FIGS. 16A and 16B show an exemplary buckle comprising a ratchet locking mechanism for the strap, according to some embodiments;

FIGS. 17A and 17B are perspective (FIG. 17A) and side (FIG. 17B) views of an exemplary buckle which is used to couple the strap to a harness, according to some embodiments;

FIG. 17C shows a strap coupled to the harness, over a body of a patient, using the exemplary buckle of FIGS. 17A and 17B, according to some embodiments;

FIGS. 18A and 18B show exemplary strap and buckles for attaching a respiration sensor to a patient's body, according to some embodiments;

FIG. 19 shows an exemplary medical device mounted on a patient's body using an attachment frame having cranes extending away from the attachment frame, according to some embodiments;

FIGS. 20A and 20B show an exemplary parallel lifting member used for lifting the medical device, in a parallel manner, from the surface of an attachment frame (also shown), according to some embodiments;

FIGS. 21A and 21B show an exemplary angular lifting member used for lifting the medical device, in an inclined manner, from the surface of an attachment frame (also shown), according to some embodiments;

FIGS. 22A and 22B show an exemplary angular aiming apparatus and an attachment frame, according to some embodiments;

FIGS. 23A and 23B show another exemplary angular aiming apparatus and an attachment frame, according to some embodiments;

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application the expression “at least one of A and B”, (e.g. wherein A and B are elements, method steps, claim limitations, etc.) is equivalent to “only A, only B, or both A and B”. In particular, the expressions “at least one of A and B”, “at least one of A or B”, “one or more of A and B”, and “one or more of A or B” are interchangeable.

In the description and claims of the application, the words “include” and “have”, and forms thereof, are not limited to members in a list with which the words may be associated.

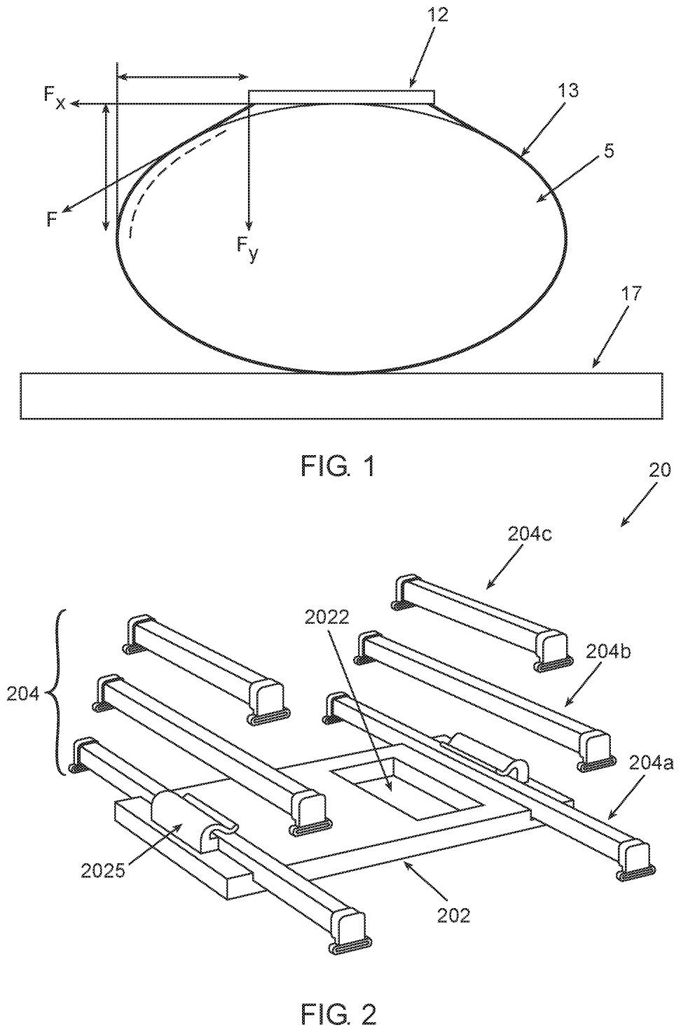

FIG. 2 shows an exemplary attachment apparatus 20 having a mediator plate 202, which is adapted for placement on the subject's body, and to which a medical device (not shown in FIG. 2), e.g., an automated insertion device, may be coupled. The mediator plate 202 may have an opening 2022 to allow access of a medical tool (e.g., needle) of the medical device coupled to the mediator plate 202, to the subject's body.

The attachment apparatus 20 may further include cranes 204, to which straps (not shown in FIG. 2) may be coupled. Using the cranes 204, which extend away, e.g., sideways, from the mediator plate 202, allows the straps to pull the mediator plate 202 in a direction substantially vertical to the plane of the mediator plate 202, thus maximizing the mounting force while minimizing the contact area of the straps with the subject's skin.

In some embodiments, the cranes 204 may be separate units removably coupleable to the mediator plate 202, using dedicated connectors 2025, e.g., latches. The cranes 204 may be coupled to one end of the mediator plate 202, or to both ends of the plate 202, as shown in FIG. 2.

In some embodiments, in order to accommodate different body types having different circumferences, as well as different locations on the subject's body having different circumferences, the attachment apparatus 20 may include a plurality of cranes 204 having varying lengths, or more then one set of cranes 204 having varying lengths, if cranes 204 are coupled to both ends of the mediator plate 202. In the example shown in FIG. 2, the mediator plate 202 is provided with three sets of cranes 204a, 204b and 204c, each set having a different length. Prior to the medical procedure, the physician can choose the crane, or the set of cranes, which is most suitable for the shape and dimensions of the specific subject's body and the specific location on the body to which the attachment apparatus 20 is to be secured.

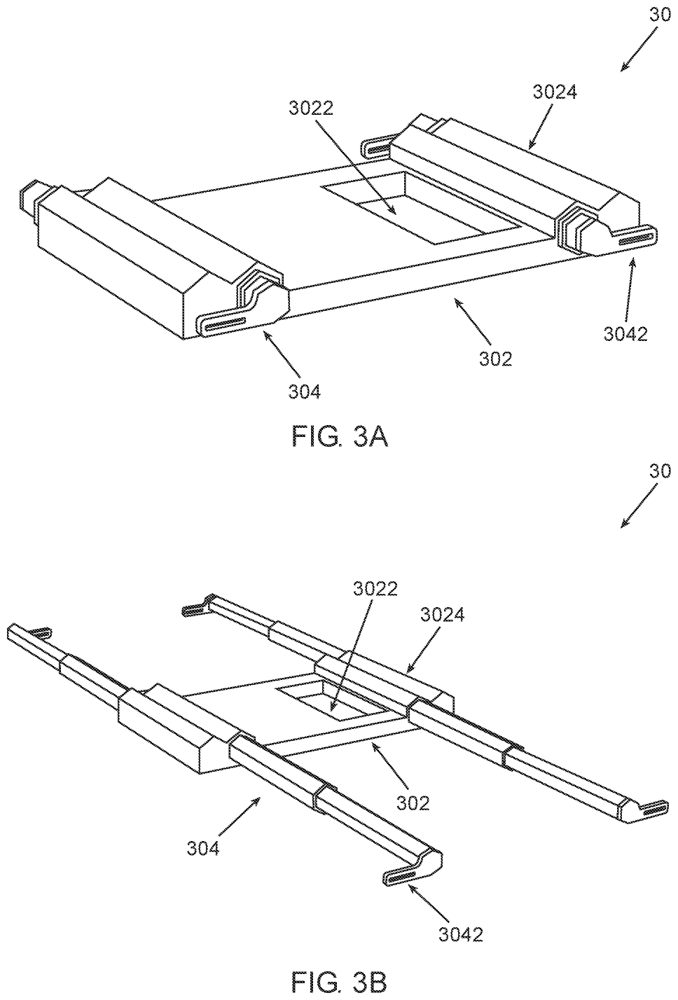

FIGS. 3A and 3B show an exemplary attachment apparatus 30 comprising a mediator plate 302 and deployable cranes 304. In some embodiments, the cranes 304 may be configured as telescopic elements. The mediator plate 302 may have an opening 3022 to allow access of the medical tool to the subject's body, and dedicated channels 3024 for accommodating the cranes 304. In some embodiments, there may be two cranes 304 provided at the end of the mediator plate 302, such that the two cranes 304 extend away from opposite sides (left/right) of the plate when deployed. Accordingly, if the mediator plate 302 is provided with cranes 304 at both of its ends, there may be four cranes altogether, with each of the four cranes being accommodated in a separate channel within the mediator plate 302, or with each pair of cranes being accommodated within a single channel at each end of the plate 302. In some embodiments, the cranes 304 may include handles 3042, which the user can grip when pulling (lengthening) the cranes 304 and pushing (shortening) the cranes 304. FIG. 3A shows the cranes 304 in a constricted (closed) state, and FIG. 3B shows the cranes 304 in an extended (open) state.

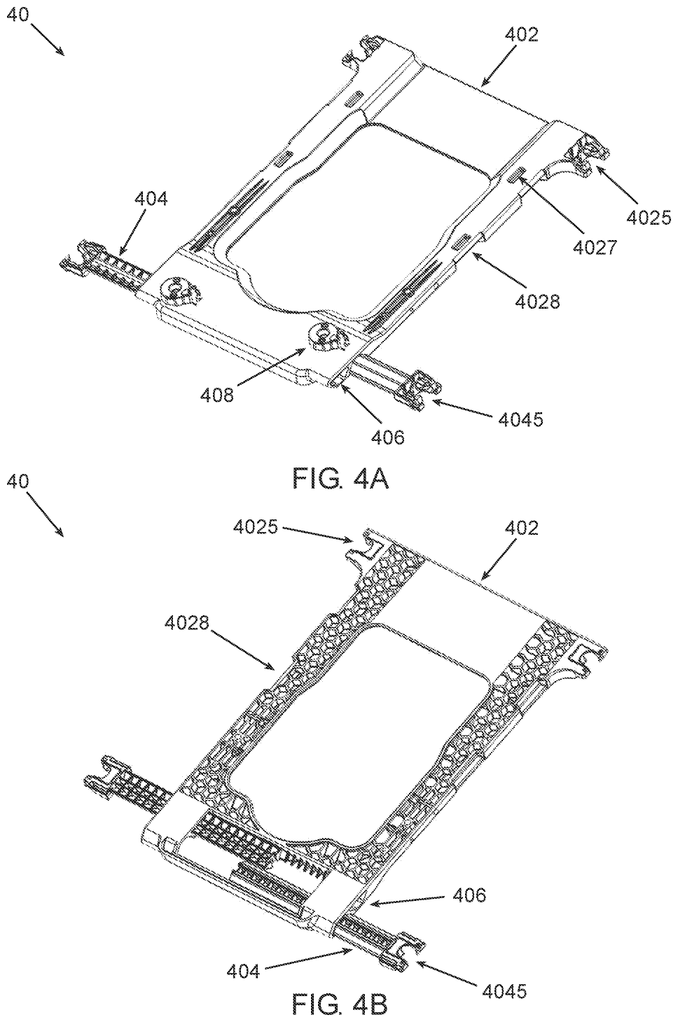

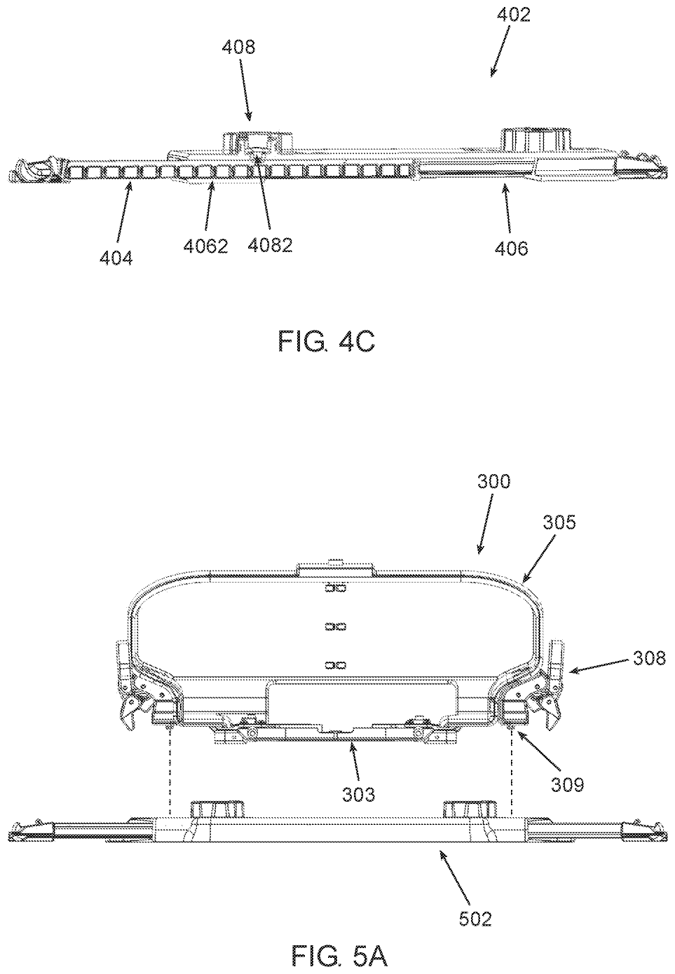

FIGS. 4A-4C show an exemplary attachment apparatus 40 comprising an attachment frame 402 and deployable cranes 404 provided at one end of the frame 402.

FIG. 4A shows a perspective top view of the attachment frame 402. The frame 402 may include a separate channel 406 for each of the cranes 404 or a single channel may accommodate both cranes 404. In some embodiments, each crane 404 may be associated with a locking knob 408, which is used for locking the position of the crane 404 within its channel 406, once it is pulled out of (or pushed into) the channel 406 to its desired position, i.e., its desired length extending away from the attachment frame 402.

In some embodiments, the distal end of the crane 404, i.e., the end which is farthest from the frame 402 when the crane is deployed, may comprise a crane connector 4045 to which a strap can be coupled, to secure the attachment frame 402 to the subject's body. In case only one end of the frame 402 is provided with cranes 404, as shown, for example, in FIG. 4A, the opposite end of the attachment frame 402 may include frame connectors 4025, to which additional straps can be coupled. It can be appreciated, that the crane connectors 4045 and the frame connectors 4025 may be either similar connectors or different types of connectors. Further, the two crane connectors 4045 may be either similar or differ from each other, and the two frame connectors 4025 may be either similar or differ from each other.

The attachment frame 402 may further include one or more grooves 4027, which are sized and shaped to receive corresponding protrusions (not shown in FIG. 4A) located on the bottom of the medical device, to facilitate and ensure alignment between the medical device and the attachment frame 402 upon positioning the medical device on the attachment frame 402. The grooves 4027, and hence the corresponding protrusions, may be distributed in an asymmetrical manner, to better ensure proper alignment between the medical device and the attachment frame 402 and prevent unintentional reverse placement of the medical device on the plate 402. In some embodiments, the bottom of the medical device may include the grooves and the attachment frame may include the corresponding protrusions.

The attachment frame 402 may further include one or more notches 4028 for receiving and engaging with corresponding one or more latches (not shown in FIG. 4A) of the medical device, to secure the medical device to the attachment frame 402 after it has been properly positioned thereon.

In some embodiments, additional elements (e.g., stoppers) may be used to ensure proper positioning of the medical device on the attachment frame, as well as to prevent movement of the medical device relative to the attachment frame after it has been positioned on the frame and before is has been secured thereto.

In some embodiments, one or more registration elements (e.g., fiducial markers) may be positioned on the attachment frame, for determining the medical device's position in the image space during image-guided procedures.

In some embodiments, the attachment apparatus is reusable, such that the same apparatus can be used repeatedly in several medical procedures, performed on different patients. In other embodiments, the attachment apparatus, or at least one or more of its components, such as the attachment frame, is disposable, such that a new apparatus (or frame) is used in each medical procedure, and the apparatus (or frame) is disposed of after the procedure has been completed.

FIG. 4B shows a perspective bottom view of the attachment frame 402 of FIG. 4A. In some embodiments, the channels 406 for accommodating the cranes 404 may be closed channels extending along the entire width of the attachment frame 402, or at least along the entire length of the cranes 404. In other embodiments, as shown in FIG. 4B, the channels 406 may be partly closed and partly open along the width of the attachment frame 402. In some embodiments, and as shown in FIG. 4B, the cranes 404 may be arranged side by side within adjacent (e.g., parallel) channels 406. In other embodiments, if the lengths of the cranes 404 is such that the cranes 404 do not overlap when both are in their constricted (closed) state, the cranes 404 may be arranged in a continuous line, accommodated within a single channel such that their proximal ends, i.e., the ends which remain within the channel/s even when the cranes are fully extended, face (and optionally abut) each other.

In some embodiments, the bottom surface of the attachment frame 402 may be covered entirely or partially with a substantially soft pad (not shown), such as a pad made of foam, to provide padding and minimize any discomfort or pain to the patient due to attachment of the attachment frame and the medical device to his/her body.

FIG. 4C shows a cross-sectional view of a crane 404 within a channel 406 of the attachment frame 402, and of a locking knob 408. In some embodiments, the locking is achieved via rotation of the knob 408. The knob 408 may include an inner projection 4082, which may be translated inwardly, toward the crane 404, as the user activates the knob 408, e.g., by rotating it. In some embodiments, rotating the locking knob 408 counter-clockwise (or clockwise) may release the projection 4082 from the crane 404, such that the crane 404 can move freely within the channel 406, and the user can adjust the length of the crane 404 which extends away from the attachment frame 402, and rotating the locking knob 408 clockwise (or counter-clockwise) may push the projection 4082 toward the crane 404, such that the projection 4082 presses the crane 404 against the back wall 4062 of the channel 406, thus locking it in place and preventing unintentional longitudinal movement of the crane 404. In some embodiments, locking may be achieved by pressing the locking knob 408 downward or sideways, or using any other suitable locking mechanism.

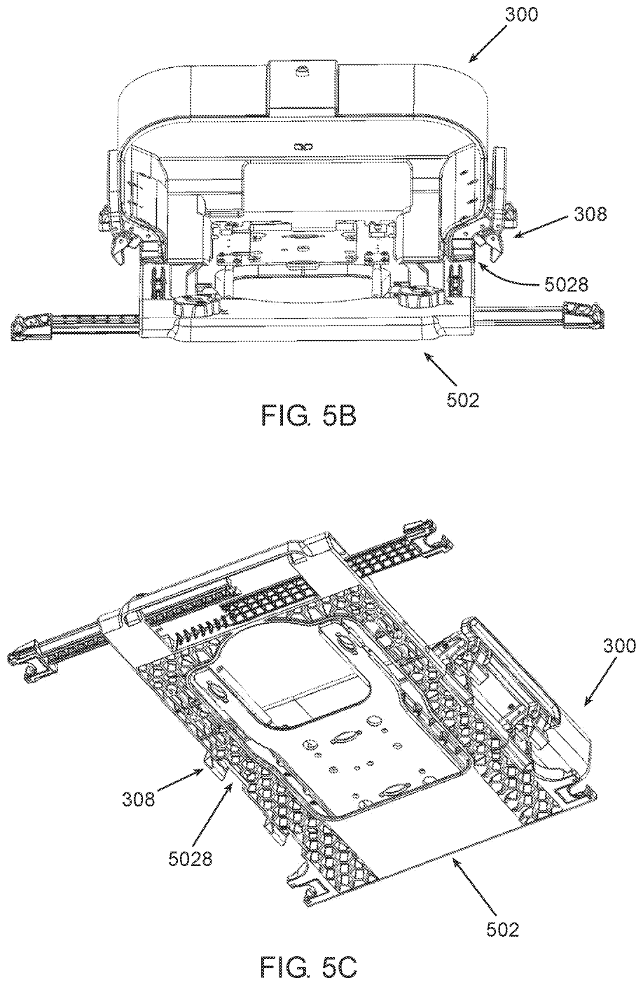

FIG. 5A shows a front view of the medical device 300 and the attachment frame 502 of FIG. 4A prior to coupling. It can be appreciated that only the base 303 and the housing (or “cover”) 305 of the medical device 300 are shown in FIG. 5A, for simplicity reasons. In some embodiments, the medical device housing 305 may include latches 308, such as two latches on opposite sides of the housing 305, to couple the device 300 to the attachment frame 502. FIG. 5A shows the latches 308 in an open state. The latches 308 may be an integral part of the device housing 305, or they may be separate units rigidly connected to the housing 305. The device housing 305 and/or the latches 308 may have protrusions 309, which are sized and shaped to be received within corresponding grooves located on the upper surface of the attachment frame, as discussed above in FIG. 4A, to facilitate and ensure alignment between the medical device 300 and the attachment frame 502 upon coupling.

FIGS. 5B and 5C show perspective front (FIG. 5B) and bottom (FIG. 5C) views of the medical device 300 and the attachment frame 502 after the medical device 300 has been placed on the attachment frame 502, with the aid of the protrusions and corresponding grooves, for example, and prior to being securely coupled thereto, i.e., prior to closing of the latches 308 onto the notches 5028 of the attachment frame 502.

In some embodiments, the medical device 300 is reusable, at least in part. In such embodiments, the medical device 300 may be positioned on the attachment frame 502 only after the medical device 300 has been covered with a sterile drape.

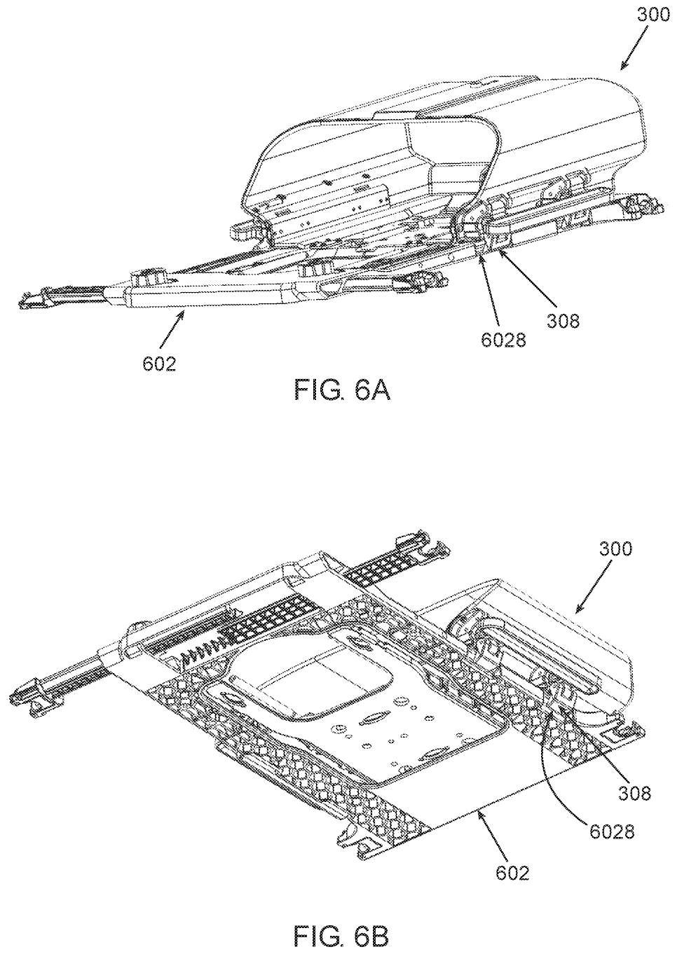

FIGS. 6A and 6B show perspective top (FIG. 6A) and bottom (FIG. 6B) views of the medical device 300 and the attachment frame 602 securely coupled, i.e., after closing of the latches 308 using the corresponding notches 6028 of the attachment frame 602.

In some embodiments, the latches 308 may include a spring which maintains them in their open position when they are not grasped by the notches 6028. Further, in some embodiments, the latches and/or the attachment frame may comprise a mechanical mechanism that prevents the user from locking the latches if the medical device in not properly positioned on the attachment frame 602, e.g., if the protrusions are not properly positioned within the grooves. For example, the latches may include a stopper (not shown) which prevents moving them downwards, and which is released only when the protrusions are inserted into the grooves, or, the notches may be initially blocked by a designated barrier, such that the barrier is removed and they can be accessed only when the protrusions are inserted into the grooves.

In some embodiments, the medical device 300 (e.g., its latches 308) and/or the attachment apparatus (e.g., the attachment frame 602) may include at least one visual or auditory indicator (not shown), to indicate to the user that the medical device 300 is properly positioned on and/or properly secured to the attachment frame 602. The indicator may be mechanically and/or electronically activated.

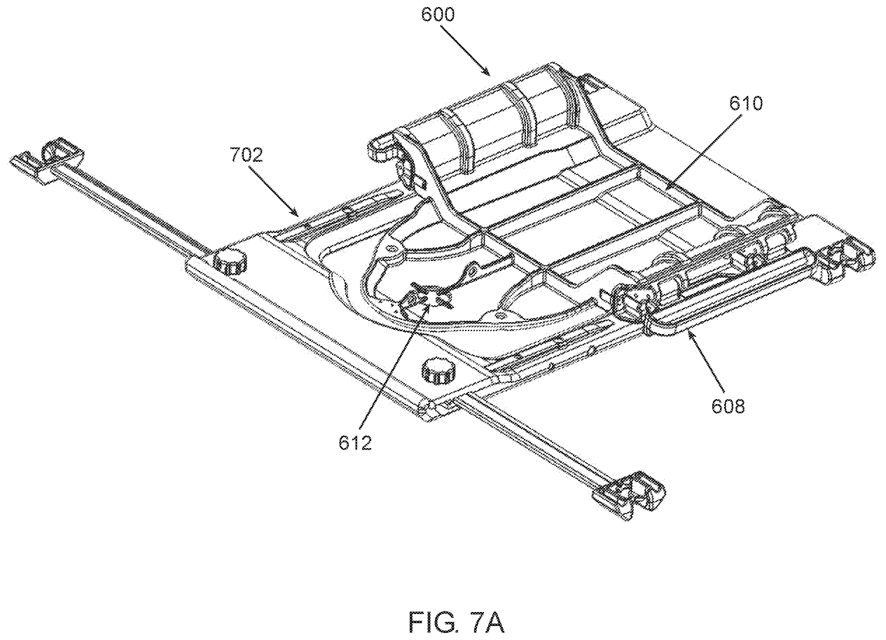

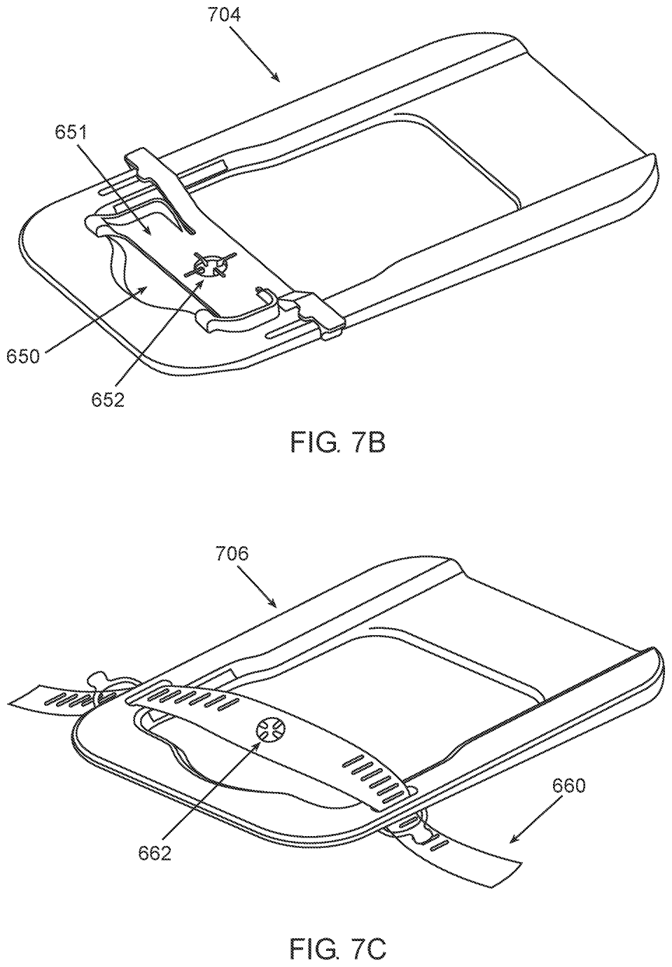

FIGS. 7A-7C show examples of an aiming apparatus (also referred to as “aiming jig” or “alignment apparatus”) coupled to an attachment frame. In some embodiments, the medical device intended to be coupled to the attachment frame is a device for inserting a medical tool, such as a needle or an introducer, into the subject's body, in order to perform a biopsy, deliver fluid to a target within the body, perform ablation, etc. Prior to inserting the medical tool into the subject's body, whether the insertion is done manually by the physician or automatically by an automated insertion device, the physician typically marks the entry point on the subject's body. Therefore, the attachment frame should be attached to the subject's body such that once the insertion device is coupled to the attachment frame, the tip of the medical tool is located directly above the entry point, or can be easily aligned with the entry point. In some embodiments, an aiming apparatus may be coupled to the attachment frame to facilitate the proper positioning of the attachment frame relative to the marked entry point. Once the proper positioning is achieved, the physician removes the aiming apparatus and couples the insertion device to the attachment frame.

FIG. 7A shows an exemplary aiming apparatus 600 which comprises a base 610 (also referred to as “plate” or “base plate”) that couples to the attachment frame 702 in a manner similar to that by which the medical device is coupled to the attachment frame, as shown in FIGS. 5A-6B above. The aiming apparatus 600 may include latches 608 which engage with the notches of the attachment frame 702, to secure the connection of the apparatus to the attachment frame. The aiming apparatus 600 may further include an opening 612 in the base 610, which is located at a location corresponding to the expected location of the tip of the medical tool relative to the attachment frame 702 when the insertion device is coupled to the attachment frame 702. The opening 612 may be provided with a cross, or any other mark, to further point to the expected location of the medical tool tip. Thus, the user places the attachment frame 702 on the subject's body, and secures the attachment frame 702 to the body using the straps, for example, while maintaining the opening 612 of the aiming apparatus 600 aligned with the entry point marked on the subject's body. Once the attachment frame 702 is securely attached to the subject's body, the user can open the latches 608 of the aiming apparatus 600, remove the aiming apparatus 600 from the attachment frame 702 and couple the insertion device to the attachment frame 702.

FIGS. 7B and 7C show alternative embodiments of an aiming apparatus. FIG. 7B shows an aiming apparatus 650 which is a narrow base 651 that is coupled to the front end of the attachment frame 704, i.e., the end close to the medical tool when the insertion device is coupled to the attachment frame 704. The narrow base includes an opening 652 for alignment with the entry point marked on the subject's body. FIG. 7C shows an aiming apparatus 660 comprising a non-flexible scale. In some embodiments, the user is required to pull both sides of the aiming apparatus 660 until the opening 662 with the cross (or other marking) of the aiming apparatus 660 is located substantially above the longitudinal medial line of the attachment frame 706.

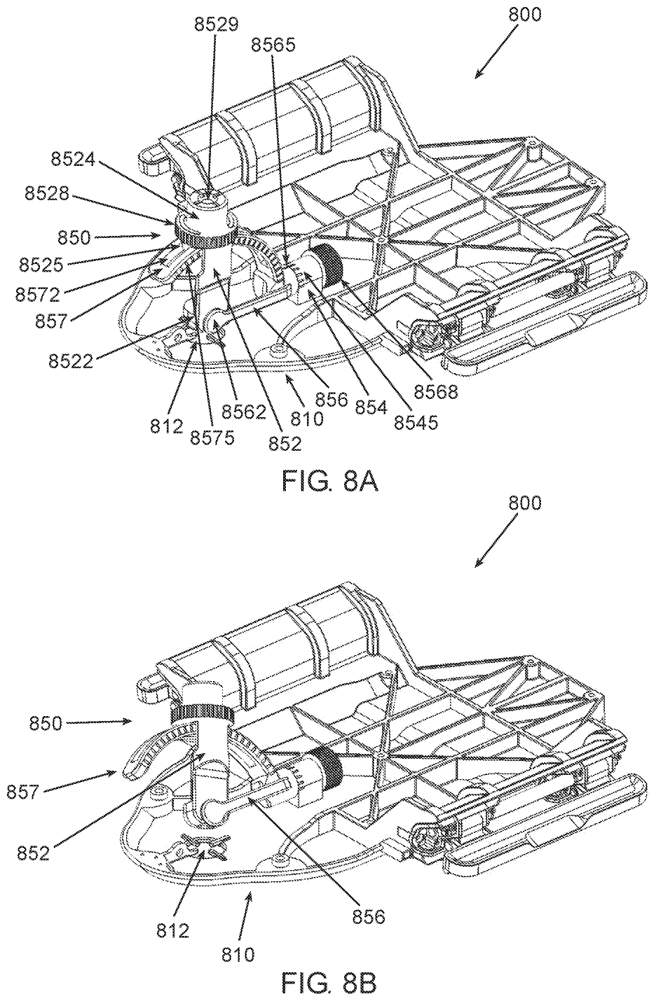

FIGS. 8A and 8B show an exemplary aiming apparatus 800, which is used not only for ensuring proper positioning of the attachment frame (not shown in FIGS. 8A and 8B), and thus of the medical device (not shown in FIGS. 8A and 8B), such that the medical tool will be aligned with the entry point, but also for ensuring proper orientation of the attachment frame, and thus of the medical device, such that the medical tool will be oriented at the entry point according to the entry angle which is required for reaching the target, according to a predetermined trajectory.

FIG. 8A shows the exemplary aiming apparatus 800 comprising an orienting mechanism 850, which may be either removably coupleable to the plate 810 or an integral part thereof. The orienting mechanism 850 may include an orienting member 852, which may simulate the medical tool or an end effector of an insertion device, such as the automated insertion device disclosed in co-owned U.S. patent application Ser. No. 16/303,536, to Arnold et al, which is incorporated herein by reference in its entirety. The orienting mechanism 850 may further include an anchoring base 854, which is coupled, either rigidly or removably, to the aiming apparatus' plate 810. In some embodiments, a rotating arm 856 may be coupled to the stationary anchoring base 854, such that the rotating arm 856 can rotate about its axis. The rotating arm 856 may include, or be coupled to, an upper arch 857 having an elongated groove 8572 extending along at least a portion of the arch's length. The upper arch 857 may be configured to rotate together with the rotating arm 856. In some embodiments, the lower portion 8522 of the orienting member 852 may be coupled to the distal end 8562 of the rotating arm 856 by means of a hinge, located above an in juxtaposition to the opening 812 of the aiming apparatus' plate 810, and the top portion 8524 of the orienting member 852 may be coupled to the arch 857 such that a protrusion 8525 of the top portion 8524 is positioned within the groove 8572. In such embodiments, the orientation of the orienting member 852 can be adjusted by rotating the rotating arm 856 about its axis, pivoting the orienting member 852 about the axis of the hinge, with the orienting member's protrusion 8525 moving within the arch's elongated groove 8572, or a combination thereof. In some embodiments, the orienting member 852 may be configured as a hollow member, the cross-section of which may be circular, oval, rectangular, or any other suitable cross-section. In other embodiments, the orienting member 852 may be configured as a cylinder, for example, with a channel formed along the length of the orienting member 852, between its top portion 8524 and its lower portion 8522, to allow viewing of the entry point, or access to the entry point, e.g., using a thin rod, a laser beam, etc., from the top portion 8524 of the orienting member 852, through the length of the orienting member 852 and through the opening 812 of the aiming apparatus' plate 810. In further embodiments, and as shown in FIGS. 8A and 8B, the top portion 8524 and lower portion 8522 of the orienting member 852 may be connected by a single wall, with the top portion 8524 having an opening 8529 and the lower portion having an opening (not shown) aligned with the opening 8529 of the top portion 8524, to allow viewing of the entry point, or access to the entry point, e.g., using a thin rod, a laser beam, etc., from the top portion 8524 of the orienting member 852, through the length of the orienting member 852 and through the opening 812 of the aiming apparatus' plate 810. It can be appreciated, that the top portion 8524 and lower portion 8522 of the orienting member 852 may be connected by more than one wall, for example two walls, three walls, four walls, etc.

In some embodiments, the orienting mechanism 850 may be configured such that the rotation angles of the rotating arm 856 and of the orienting member 852 relative to the rotating arm 856, are limited to the maximal angles which can be reached by the insertion device, e.g., by the end effector of the insertion device. Thus, in case an attempt to align the attachment frame, using the aiming apparatus 800, according to the desired insertion angle, fails, the user can adjust the position and/or the angle of the attachment frame relative to the patient's body, e.g., using cushion/s or a lifting member, as disclosed hereinbelow.

In some embodiments, the anchoring base 854 may be provided with notches 8545, and the proximal end of the rotating arm 856 may be provided with an indicator 8565, which is positioned adjacent the notches 8545, to indicate to the user, and assist him/her in controlling, the degree of rotation of the rotating arm 856. In some embodiments, the arch 857 may be provided with notches 8575 disposed along at least a portion of the arch 857, to indicate to the user, and assist him/her in controlling, the degree of pivoting of the orienting member 852 relative to the rotating arm 856.

In some embodiments, once the desired orientation of the orienting member 852 has been reached, the user can lock the orienting member 852 at the desired orientation, using one or more locking mechanisms. In some embodiments, the rotating arm 856 may comprise a knob 8568, which can be rotated, for example clockwise, to lock the orientation of the rotating arm 856 via friction with the anchoring base 854, and prevent further rotation of the rotating arm 856 about its axis. Similarly, in some embodiments, the orienting member 852 may comprise a knob 8528, which can be rotated, for example clockwise, to lock the orientation of the orienting member 852 relative to the rotating arm 856, i.e., by locking the position of the protrusion 8525 within the elongated groove 8572, via friction with the arch 857, and prevent further movement of the orienting member 852 along the arch 857. FIG. 8B shows the aiming apparatus 800 after the orienting member 852 has been set in the desired orientation relative to the opening 812 of the plate 810.

In some embodiments, after the attachment frame has been secured to the patient's body in alignment with the entry point and at the desired entry angle, and while the aiming apparatus 800 is still coupled to the attachment frame, imaging of the region of interest, which includes both the entry point and the target point, may be initiated, to verify, prior to removal of the aiming apparatus 800 from the attachment frame and coupling of the insertion device to the attachment frame, that the set entry angle matches the desired entry angle according to the preplanned trajectory between the entry point and the target.

In some embodiments, the orienting member 852 may include one or more registration elements, e.g., fiducial markers, such that the position of the orienting member 852 relative to the image space can be determined. In some embodiments, the determined position of the orienting member 852 relative to the image space can then be used, for example by the insertion device's software, in order to position the medical tool, or the insertion device's end effector to which the medical tool is coupled, in the desired position and orientation for the commencement of the medical procedure.

In some embodiments, the attachment frame may include a plurality of visible markings, e.g., notches, grid lines, to assist the user if repositioning of the attachment frame is required. The markings may be configured such that they can be detected also by the imaging system, e.g., in a CT image.

It can be appreciated, that alternative orienting mechanisms may be used with the attachment frame. For example, in some embodiments, a ball and socket mechanism (not shown) may be utilized, e.g., a ball having a hollow channel formed therethrough (a bead-like configuration) may be coupled to the opening 812 of the aiming apparatus' plate 810, such that it can be rotated therein and locked at the desired orientation.

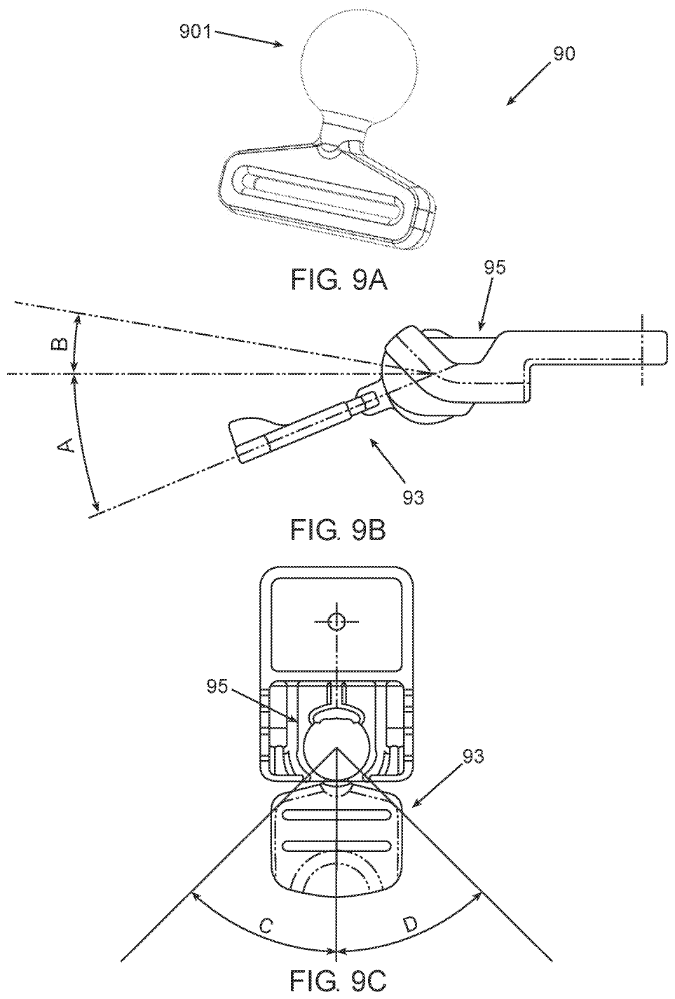

FIG. 9A shows an exemplary buckle 90 used for connecting the strap to the crane and/or to the attachment frame. In some embodiments, the connection between the buckle 90 and the connector at the distal end of the crane and/or the connector at the end of the attachment frame opposite the end to which the cranes are coupled, may be a ball-and-socket connection, such that the crane connector/attachment frame connector is configured as a socket and the buckle connector 901 is configured as a ball (or vice versa). Such buckles may be referred to as “ball buckles”. In some embodiments, the socket connector may include a rigid outer structure and a softer inner portion, to allow the ball (male) connector 901 to be snapped into the socket.

FIGS. 9B and 9C demonstrate optional rotation angles of an exemplary ball buckle 93 after connection to a socket connector 95. Using a ball-and-socket connection mechanism between the straps and the attachment frame/medical device provides the straps with a wide rotation range, which provides a much-desired flexibility in the location and orientation of the attachment frame and/or the medical device, on the patient's body. In some embodiments, the rotation range of the ball buckle 93 about the longitudinal axis of the socket connector 95 may be, for example, downward rotation range “A” between 0-120 degrees and upward rotation range “B” between 0-5 degrees. It can be appreciated that the rotation ranges about the longitudinal axis of the socket connector 95 may comprise any smaller or larger ranges of values. In some embodiments, the upward rotation range “B” may be dictated by the rotation angle which is set to be the angle at which the ball buckle 93 disconnects from the socket connector 95. In some embodiments, the rotation range of the ball buckle 93 about the longitudinal axis of the ball buckle 93 may be symmetrical, for example, the left rotation range “C” and the right rotation range “D” may both be between 0-45 degrees. It can be appreciated that the rotation ranges about the longitudinal axis of the ball buckle 93 may be asymmetrical, and they may comprise any smaller or larger ranges of values.

It can be appreciated that other connection mechanisms, which provide similar, or substantially similar, rotation/pivoting capabilities for the straps, may be equally advantageous.

As the patient inhales, his/her lungs inflate, causing the chest/abdomen to expand radially. As the patient exhales, his/her lungs deflate, causing the chest/abdomen to shrink radially. Thus, if the straps which secure the medical device or the attachment frame to the patient's body have no flexibility, there is a risk that they might become too tight during inhalation, which might cause significant discomfort to the patient, and/or that they become too loose during exhalation, which might enable relative movement between the medical device and the patient's body. Such relative movement might cause not only significant discomfort to the patient, but also physical harm to the patient, such as tearing of tissues due to uncontrolled sudden movements of the medical tool while it is positioned inside the patient's body. Further, in some insertion devices, steering of the medical tool is based on the assumption that there is no relative movement between the device and the patient's body, thus, relative movement between the device and the patient's body might impair the accuracy of the device and compromise the success of the medical procedure.

In order to accommodate body movements associated, for example, with breathing, and to ensure that the straps do not become too tight during inhalation and/or too loose during exhalation, in some embodiments, the buckles coupling the straps to the attachment frame/medical device may include, or be coupled to, a stretching mechanism, e.g., having an elastic member, which can stretch during inhalation and return to its relaxed state during exhalation. In other embodiments, the buckle itself may be formed having elastic capabilities, as described below.

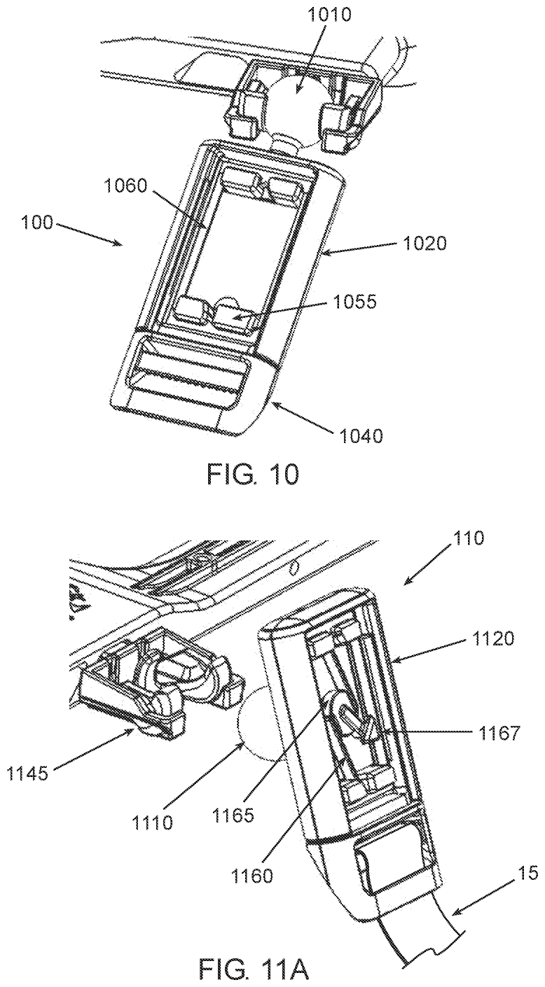

FIG. 10 shows a buckle 100 comprised of two parts which are moveable relative to each other; a first (upper) part 1020, which includes the connector 1010 (e.g., ball connector), that can be coupled to the connector (e.g., socket connector) of the crane or of the attachment frame, and a second (lower) part 1040, to which the strap is coupled. Each of the upper and lower parts of the buckle 100 may include anchor/s 1055 which hold an elastic member 1060, e.g., a silicone band, such that the upper portion of the elastic member is secured to the upper part 1020 of the buckle 100, and the lower portion of the elastic member is secured to the lower part 1040 of the buckle 100. Thus, as the strap is being pulled by the user during securing of the attachment frame to the patient's body and/or by the patient's body during inhalation or coughing, for example, the lower part of the buckle is pulled together with the strap, resulting in stretching of the elastic member 1060 and movement of the lower part 1040 of the buckle, together with the strap, away from the upper part 1020 of the buckle.

In some embodiments, the user may wish to verify, during strapping of the attachment frame to the patient's body, that the attachment enables both stretching and relaxing of the elastic member, which are required to accommodate the movements of the patient's body during the breathing cycle, as well as coughing, etc.

For this purpose, the buckle 100 may include an indicator, which may be associated with the elastic member 1060, and indicates to the user if the attachment frame is attached such that it enables both stretching and relaxing of the elastic member. For example, the upper part 1020 of the buckle may include an indication bar provided with a dial, which moves along the indication bar as the elastic member 1060 stretches and relaxes. The indication bar may comprise a color scale. For example, it may include a middle green section located between two red sections, such that the dial being on (or pointing to) the green section indicates that the degree of stretching of the elastic member is in the range which allows stretching and relaxing of the elastic member 960 during the breathing cycle.

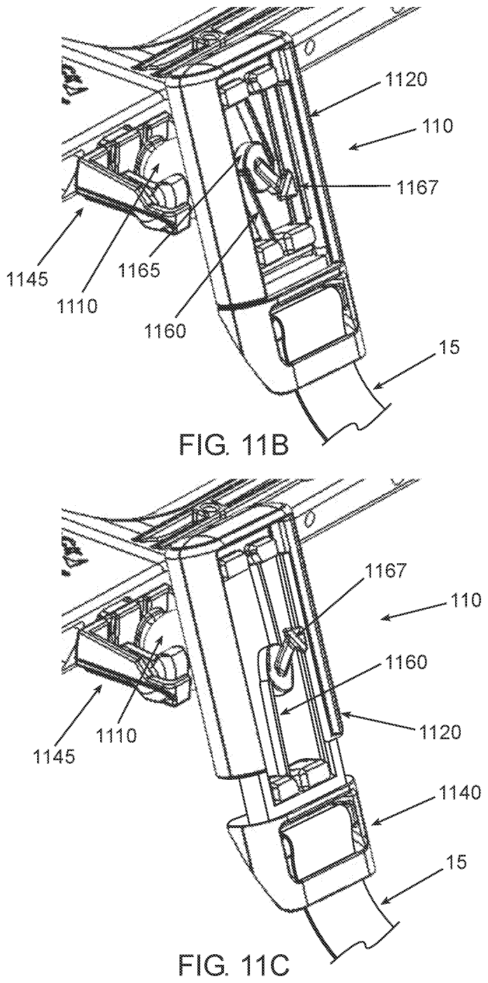

FIGS. 11A-11C show an exemplary buckle 110 having an alternative indicator. The buckle 110 may include an elastic member 1160 that includes an integral rotating member 1165, which rotates as the elastic member 1160 is being stretched and relaxed. Alternatively, the elastic member 1160 may be wound around a rotating element, which is rotated by the elastic member as it is being stretched and relaxed. The rotating element 1165 may be provided with an arrow 1167, or simply a rod, which can indicate as to the degree of stretching of the elastic member 1160, such that as the user pulls the strap to tighten its grip around the patient's body, he/she can aim for the desired tightening degree which will enable both stretching and relaxing of the elastic member 1160 while the attachment frame, and the coupled medical device, are secured to the patient's body.

FIG. 11A shows the ball buckle 110 with the coupled strap 15 prior to connection to the socket connector 1145 of the crane or attachment frame. In some embodiments, the ball connector 1110 is located at the back side of the upper part 1120 of the buckle 110, i.e., at the side facing the crane or the attachment frame. FIG. 11B shows the ball buckle 110 with the coupled strap 15 connected to the socket connector 1145, with the elastic member 1160 of the buckle 110 being in its relaxed (unstretched) state. The relaxed (unstretched) state may be indicated by the arrow 1167 pointing upwards and to the right, for example. FIG. 11C shows the ball buckle 110 with the strap 15 connected to the socket connector 1145, with the elastic member 1160 of the buckle 110 being in its stretched state, resulting from the pulling of the strap 15 and the lower part 1140 of the buckle to which it is coupled away from the upper part 1120 of the buckle. The stretched state may be indicated by the arrow 1167 pointing downwards and to the right, for example.

In some embodiments, the upper part of the buckle may be provided with a scale (not shown), such as a numeric scale, or a color scale, to which the arrow may point, to assist the user in identifying the degree of stretching of the elastic member. For example, the upper part of the buckle may include a strip having a green section in between two red sections, such that the arrow pointing to the green section indicates that the degree of stretching of the elastic member is in the required range.

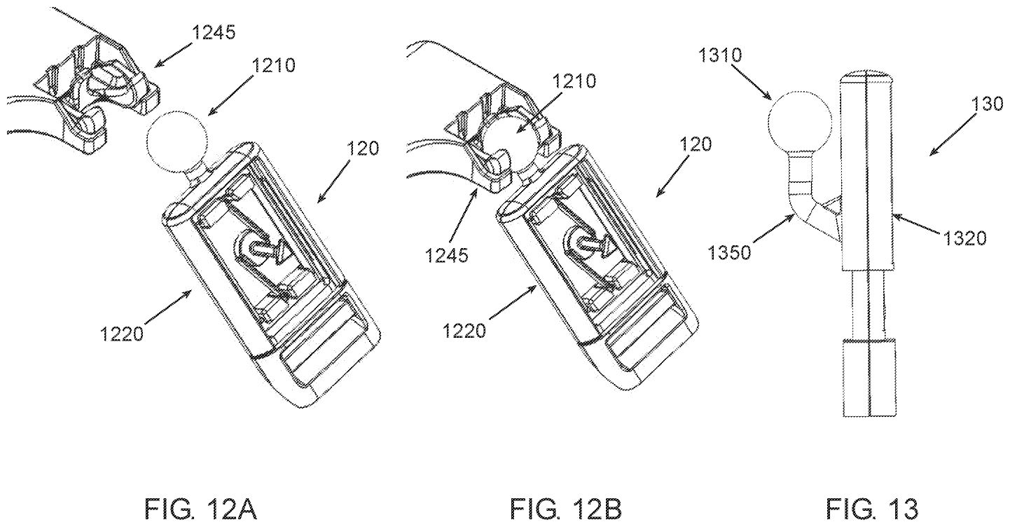

FIGS. 12A and 12B show a ball buckle 120 similar to the ball buckle of FIGS. 11A-11D, except that the ball connector 1210 is located at the top of the upper part 1220 of the ball buckle 120 instead of at its back side. FIG. 12A shows the ball buckle 120 prior to connection to the socket connector 1245. FIG. 12B shows the ball buckle 120 connected to the socket connector 1245.

FIG. 13 shows a side view of another exemplary ball buckle 130, in which the ball connector 1310 is coupled to the upper part 1320 of the buckle 130 by means of an arm, in this case, an angled arm 1350. The upper part of the buckle 1320, the angled arm 1350 and the ball connector 1310 may be an integral unit, or they may be individually manufactured and then assembled together.

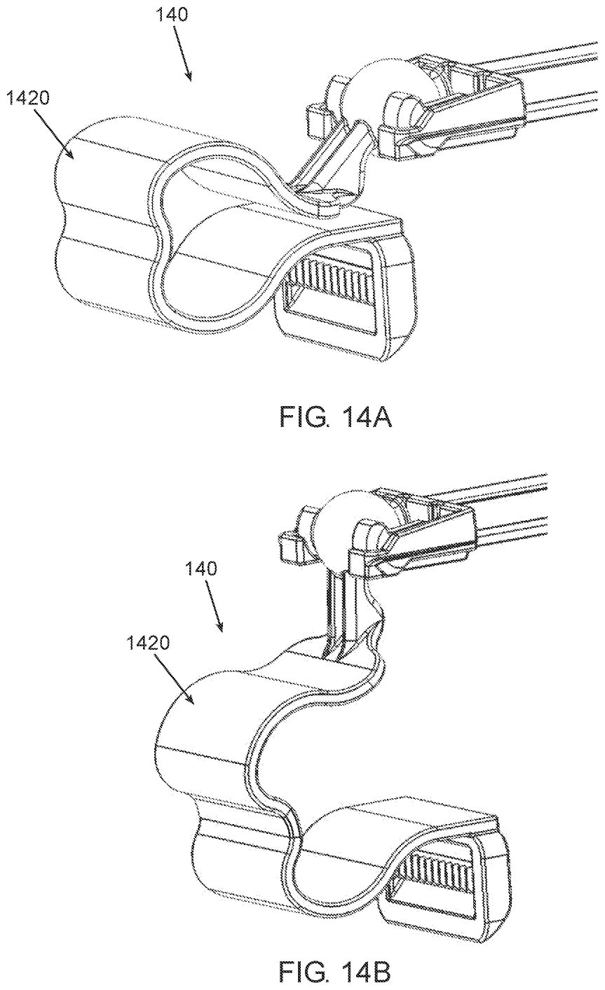

FIGS. 14A and 14B show an exemplary buckle 140 having elastic capabilities, due to its geometry and the material/s it is fabricated of, thus obviating the need for a separate stretching mechanism. In some embodiments, the buckle 140 may be configured essentially as a flat spring or a leaf spring. The buckle 140 may comprise a curved strip 1420 that can be stretched to a predesigned degree, to enable lengthening of the coupled buckle and strap by a predetermined length, and once the tension (pulling) is removed, the buckle returns to its relaxed state. FIG. 14A shows a perspective view of such a stretchable buckle 140 in its relaxed state. FIG. 14B shows a perspective view of the stretchable buckle 140 in its stretched state. The stretchable buckle may be made of springy steel material, such as stainless steel, or of a nylon polymer, or any other suitable material.

It can be appreciated that the stretching mechanisms and stretchable buckles disclosed hereinabove are not limited to implementation in buckles having a ball connector, and they may be implemented in buckles having any other type of suitable connector for coupling them to the crane and/or attachment frame and/or medical device.

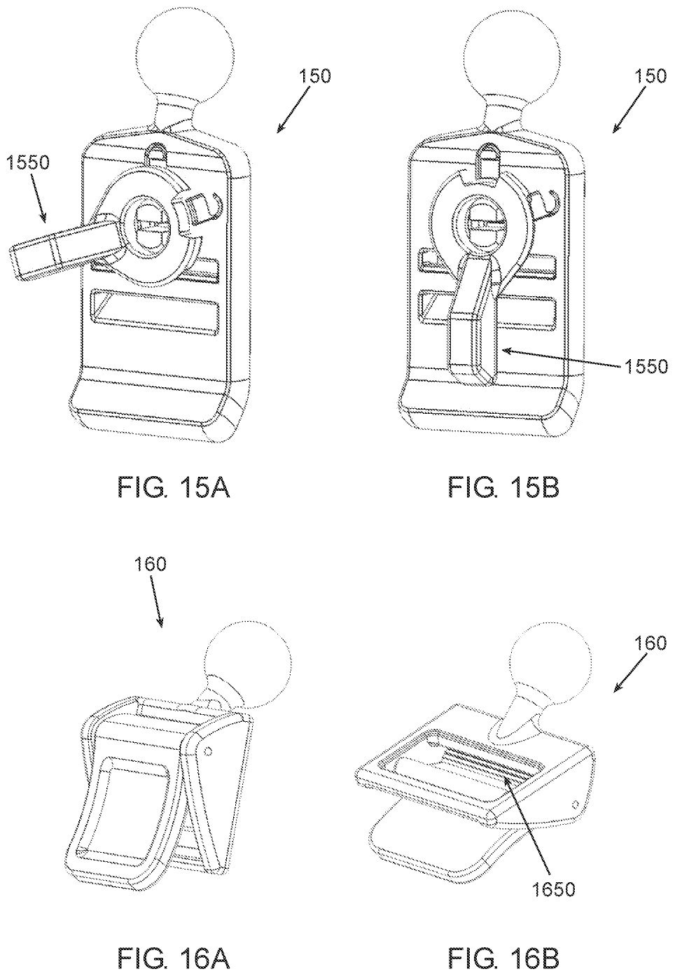

FIGS. 15A and 15B show an exemplary buckle 150 having a locking mechanism which prevents movement of the strap (not shown in FIGS. 15A and 15B) relative to the buckle 150 once the attachment frame is properly secured to the patient's body. In some embodiments, the buckle 150 may include a locking handle 1550, which can be moved by the user from an unlocked state (FIG. 15A) to a locked state (FIG. 15B), and vice versa. In some embodiments, rotating the handle 1550 counterclockwise may result in the handle 1550 applying pressure on the strap, such that it is slightly pressed against the buckle 150 and thus cannot be unintentionally released or loosened. Rotating the handle 1550 clockwise, in such embodiments, may release the handle 1550 from the strap, allowing the user to stretch and/or release the strap, as needed. In alternative embodiments, locking may be achieved by rotating the handle 1550 clockwise and unlocking may be achieved by rotating the handle 1550 counterclockwise. In some embodiments, as shown in FIGS. 15A and 15B, the buckle 150 may include markings to indicate the locked and unlocked states.

It can be appreciated that other locking mechanisms may be implemented in the buckles. For example, FIGS. 16A and 16B show an exemplary buckle 160 having a locking mechanism which has a ratchet 1650 and locks the strap in place by means of friction. Another example may be a locking mechanism based on puncturing the strap, i.e., the buckle may be provided with a tack-like element, which the user can press such that the tip punctures the strap and affixes the strap to the buckle.

Further, it can be appreciated that the locking mechanisms disclosed hereinabove are not limited to implementation in buckles having ball connectors, and they may be implemented in buckles having any other type of suitable connector for coupling them to the crane and/or attachment frame and/or medical device.

It can be appreciated that all of the buckles disclosed hereinabove may be used for securing either an attachment frame to the patient's body or a medical device directly to the patient's body, when an attachment frame is not employed. In the latter case, the buckles may be coupled to connectors, such as socket connectors, provided on the medical device itself, e.g., on the base or the housing/cover of the medical device.

In some embodiments, the attachment apparatus may include a harness (also referred to as “back base”), which is positioned between the patient's body and the patient bed. The harness may be either reusable, i.e., used in multiple procedures, or disposable, i.e., disposed of after a single use.

In some embodiments, the straps of the attachment apparatus may be rigidly connected to the harness such that their loose ends comprising the connectors, e.g., ball buckles, are removably coupleable to the attachment frame or directly to the medical device. In other embodiments, the straps may be rigidly connected to the attachment frame or to the medical device such that their loose ends comprising the connectors are removably coupleable to the harness. In further embodiments, the straps may be stand-alone components, initially unattached to either of the harness or the medical device/attachment frame, such that the user connects the straps to both the harness and the attachment frame/medical device during preparation for the medical procedure.

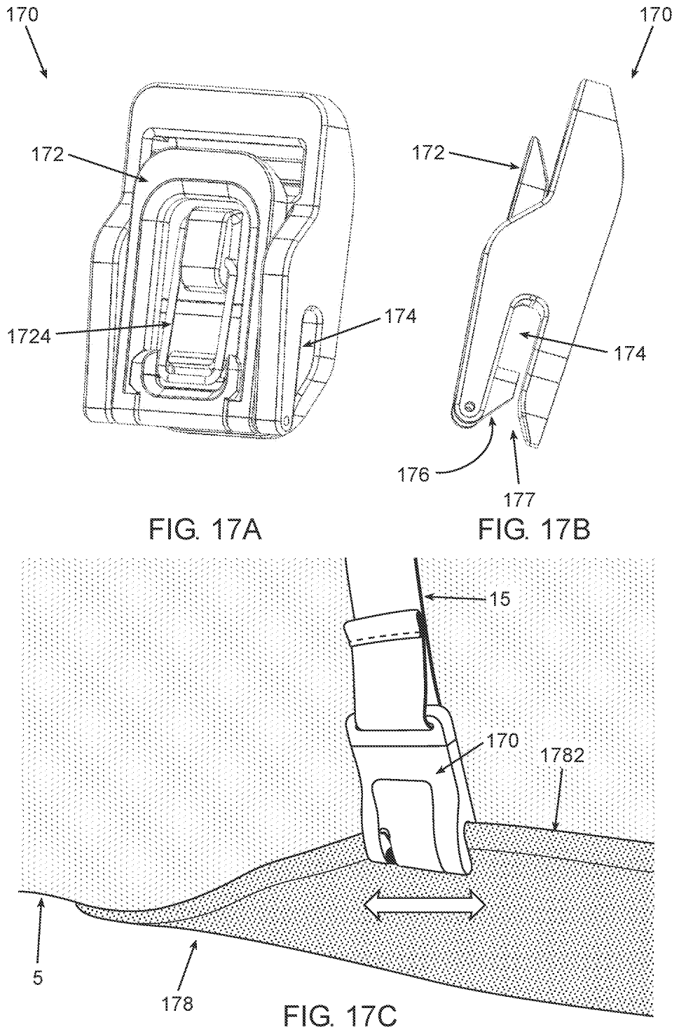

FIGS. 17A and 17B show perspective (FIG. 17A) and side (FIG. 17B) views of an exemplary buckle 170 for coupling the strap/s to the harness (not shown in FIGS. 17A and 17B). The buckle 170 may be configured as a clip having a lever 172, and an inner space 174 with at least one tooth 176 for gripping the harness, once it has been received within the space 174. Lifting the lever 172 causes upward movement of the tooth 176, i.e., away from the inner space 174, and lowering the lever 172 causes downward movement of the tooth 176, i.e., into the inner space 174, or vice versa. Lowering of the lever 172 may be done manually, or the lever 172 may include a spring 1724, as shown in FIG. 17A, such that when the user releases the lever, the tooth 176 automatically moves back into the inner space 174 and grips the harness. In some embodiments, the user may be required to lift the lever 172 in order to position the edge of the harness within the inner space 174 of the buckle 170, whereas, in other embodiments, the entry to the inner space 174 may be defined by inclined surfaces of the buckle wall 177 and/or of the tooth 176, allowing the user to couple the buckle 170 to the harness by positioning the edge of the harness at the entry to the inner space 174 and then slightly pushing the buckle 170 toward the harness, to cause the edge of the harness to move into the inner space 174 and be gripped by the tooth 176.

In some embodiments, removal of the strap from the harness is achieved by lifting the lever 172, so as to release the tooth's grip on the harness.

FIG. 17C shows the buckle 170 attached to the harness 178, over the patient's body 5 (e.g., the torso, the waist). In some embodiments, once the buckle 170 is attached to the harness 178, it can be moved sideways along the edge 1782 of the harness 178, as shown in FIG. 17C, allowing adjustment of the location of the straps 15 along the patient's body 5. In some embodiments, the edge 1782 of the harness 178 is defined by a wire disposed along the circumference of the harness 178. In some embodiments, the wire may be coated by a fabric (e.g., a keder).

In some embodiments, the buckle which secures the strap to the harness may be rigidly coupled to the harness, i.e., an integral component of the harness, separate from the strap, such that securing the strap to the harness is achieved by coupling the strap to the harness' buckle.

In some embodiments, the buckles used for coupling the straps to the harness may include, or be coupled to, a stretching mechanism similar to the any one of the stretching mechanisms described hereinabove with regard to the buckles used for coupling the straps to the attachment frame or to the medical device, or any other suitable stretching mechanism.

In some embodiments, once the attachment frame is secured to the patient's body, the user may wish to check the attachment force to verify that it is equal to or larger than the insertion force, such that inserting the medical tool into the patient's body will not cause movement of the attachment frame with the medical device coupled thereto relative to the patient's body. The attachment force may be measured using standard force measurement instruments or using a dedicated force measurement jig, having a mechanical or electronic indicator to indicate to the user once the required force (e.g., 1 Kg, 1.5 Kg, etc.) is reached. The attachment force may be measured relative to the attachment frame or relative to the alignment jig when coupled to the attachment frame.

In some embodiments, the attachment apparatus is provided with, or is adapted to have coupled thereto, a respiration sensor. The respiration sensor may be a pressure sensor or a sensor which senses the chest or abdominal expansion/contraction, and provides the physician with data regarding the breathing cycle of the subject, such as the respiration waveform.

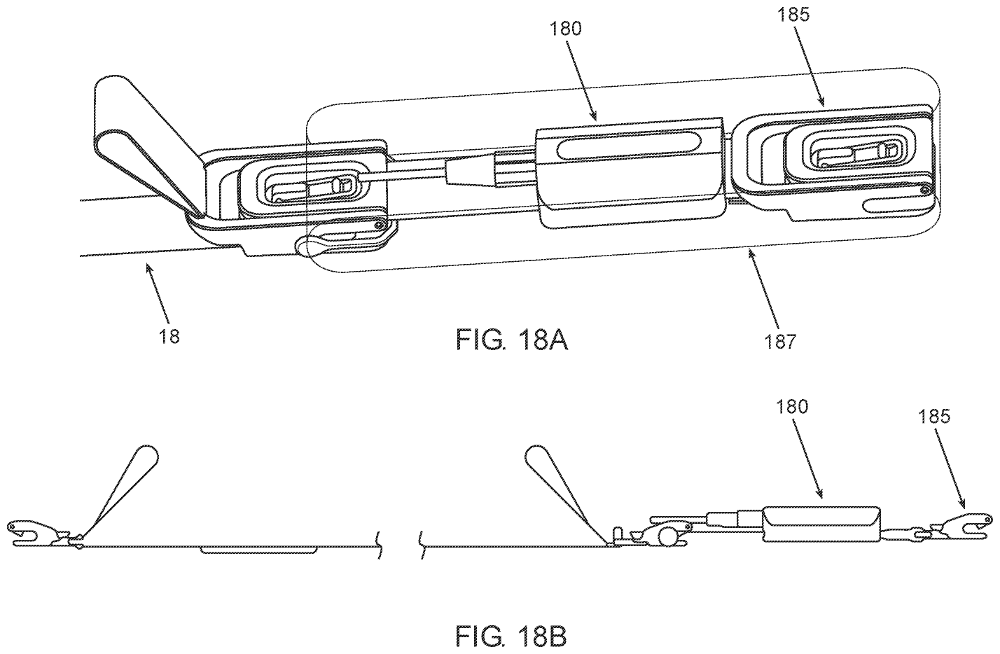

In some embodiments, as shown in FIGS. 18A and 18B, a respiration sensor 180 may be provided separately from the attachment apparatus, coupled to a separate strap 18. The strap 18 may be coupleable to the harness on both sides of the patient using connectors 185, which may be similar to the buckles shown in FIGS. 17A and 17B. In other embodiments, the respiration sensor may be coupled to one of the straps used for securing the attachment frame to the patient's body. In some embodiments, the respiration sensor 180 and at least the connector 185 by which it is attached to the harness, may be covered by a designated drape 187.

The data from the respiration sensor 180 may be transferred to the system controller (not shown) and may be used for synchronizing the activation of the medical device (e.g., the insertion and/or steering of a needle) and/or the initiation of a scan (e.g., a CT scan) in image-guided procedures, with a specific point or points in the breathing cycle. The initiation of the scan may be automatic, or the user may be prompted to manually initiate a scan. Respiration sensors which may be utilized are, for example, Respiration Sensor-SA9311M manufactured by Thought Technology Ltd. of Montreal West, Quebec, Canada, or the Piezoelectric Respiration (PZT) Sensor manufactured by Plux of Lisbon, Portugal.

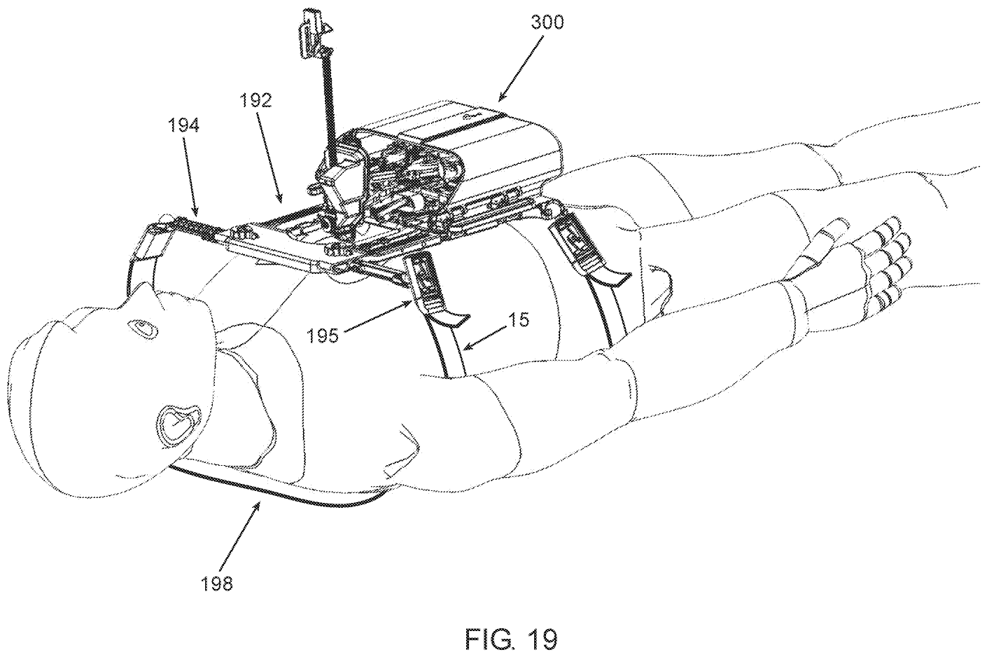

FIG. 19 shows a medical device 300 mounted on the patient's body using an attachment frame 192 having cranes 194 extending away from the attachment frame 192, as disclosed hereinabove. The medical device 300 may be an automated insertion device, as disclosed, for example, in the abovementioned U.S. patent application Ser. No. 16/303,536. The attachment frame 192 may be secured to the patient's body using straps 15 coupled to the attachment frame/cranes, using ball buckles 195, at one end, and to the harness 198 at the opposite end.

In some embodiments, the attachment frame and/or the medical device may comprise a sensor to indicate if, after the attachment frame has been properly secured to the patient's body and/or after the medical device has been properly coupled to the secured attachment frame, there is movement of the attachment frame and/or the coupled medical device relative to the patient's body, above a predetermined threshold. The sensor may be, for example, a distance sensor, a proximity sensor, an accelerometer (the signals of which may be compared to those of an accelerometer positioned on the patient's body), etc. The relative movement indication provided by the sensor may be visual or auditory.

In some embodiments, due to the natural curves of the patient's body, the attachment frame may require a slight lifting and/or angling, in order to stabilize the medical device when it is attached to the body. The attachment apparatus may include, in such embodiments, one or more cushions (not shown), which can be placed underneath the medical device and/or the attachment frame and/or the straps, to ensure stable positioning of the medical device, using the attachment frame, on a curved surface of the patient's body. The cushions may further be used for adjusting the positioning angle of the attachment frame relative to the patient's body, to ensure that the medical device, e.g., and insertion device, can be positioned relative to the body such that a desired insertion angle for the medical tool can be reached. The cushions may further provide padding and minimize any discomfort or pain to the patient due to attachment of the attachment frame and the medical device to his/her body. In some embodiments, the cushions may be inflatable. In some embodiments, the cushion may be filled, at least in part, with granules, either natural or artificial, such that when vacuum is applied to the cushion, the granules are pressed against each other and the cushion stiffens, providing stability to the medical device, and ensuring that there is substantially no relative movement between the medical device and the patient's body. In some embodiments, the cushion may include therein (or thereon) one or more registration elements (e.g., fiducial markers), for determining the medical device's position in the image space during image-guided procedures, in case the medical device itself is positioned outside the scanned volume, as disclosed, for example, in co-owned U.S. Patent Application Publication No. 2018/368,922, to Roth et al.

In some embodiments, the attachment frame may be configured such that its shape, more specifically, the shape of its bottom surface, i.e., the surface which contacts the patient's body, can adjust according to the natural curves of the patient's body. Alternatively, the user may be provided with a plurality of attachments frames, having a variety of shapes and/or dimensions, which the user can choose from, such that the attachment frame used would be the most suitable for the contour of the patient's body in the location to which the attachment frame is to be secured.

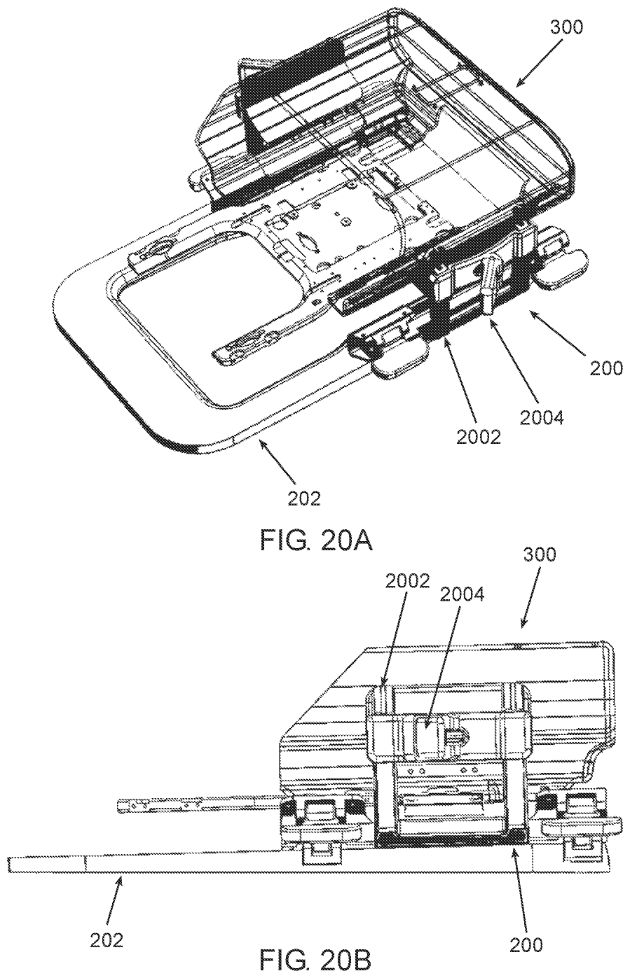

In some embodiments, as shown in FIGS. 20A and 20B, the attachment apparatus may include a parallel lifting member 200, which enables the user to lift the medical device 300, in a parallel manner, from the surface of the attachment frame 202, and thus from the surface of the patient's body, if desired due to the body's contour at the location of attachment. The lifting member 200 may include one or more rails 2002, along which the medical device can be moved, and a fixator 2004, to fixate the position of the medical device 300 along the rail 2002, once the desired height above the attachment frame 202 has been reached.

The parallel lifting member 200 may be rigidly coupled to, or an integral part of, the attachment frame 202 or the base/housing of the medical device. Alternatively, the parallel lifting member 200 may be a separate unit which is removably coupleable to both the attachment frame 202 and the medical device 300, such that the user can decide, while securing the attachment frame 202 to the patient's body, if the lifting member 200 is required or not.

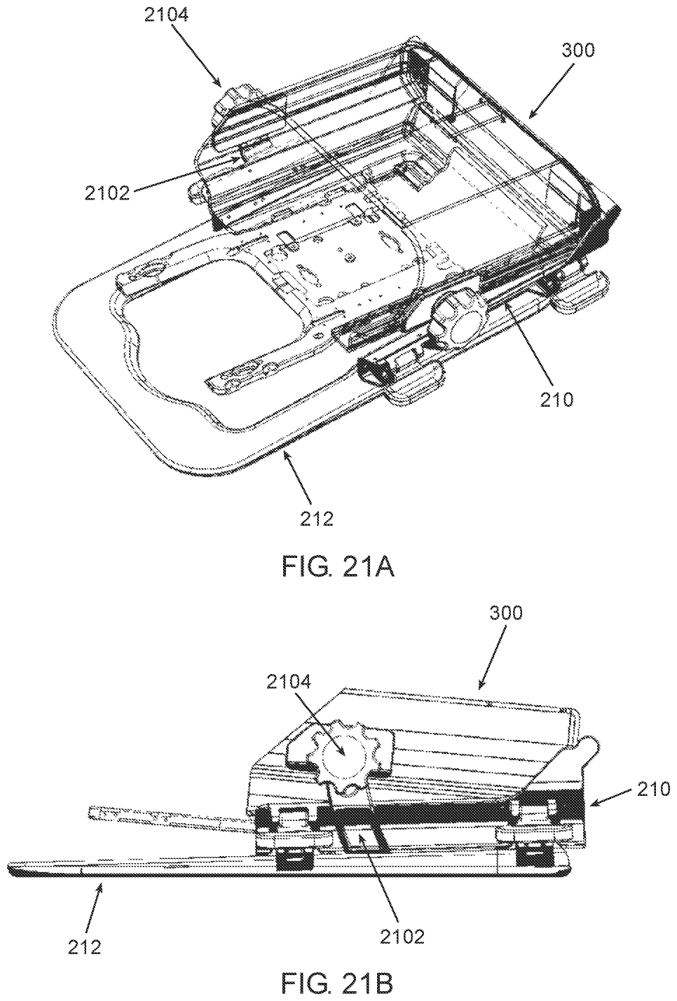

In some embodiments, as shown in FIGS. 21A and 21B, the attachment apparatus may include at least one angular lifting member 210, which enables the user to lift the medical device, in an angular (or—inclined) manner, from the surface of the attachment frame 212, and thus from the surface of the patient's body. The angular lifting member may include a curved rail 2102, along which the medical device 300 can be moved, and a fixator 2104, to fixate the position of the medical device 300 along the rail 2102, once the desired angle of the device relative to the body surface has been reached. In some embodiments, as shown in FIG. 21A, the attachment apparatus may include two curved rails 2102, positioned on opposite sides of the attachment frame 212, optionally in a symmetrical manner. The curved rails 2102 may be positioned close to the front end of the medical device, to allow lifting of the front portion of the medical device 300, as shown in FIGS. 21A and 21B, or they may be positioned close to the rear end of the medical device (not shown), to allow lifting of the rear portion of the medical device 300. In some embodiments, not shown, one or more curved rails 2102 may be positioned on only one side of the attachment frame 212, to enable tilting of the medical device 300 toward the opposite side of the attachment frame 212. It can be appreciated, that the curved rail/s 2102 may be positioned in any other applicable manner, to enable any desired tilting of the medical device 300 relative to the attachment frame 212. Further, the attachment apparatus may include a mechanism which enables simultaneous lifting and tilting of the medical device 300 relative to the attachment frame 212.

The angular lifting member 210 may be rigidly coupled to, or an integral part of, the attachment frame 212 or the base/housing of the medical device. Alternatively, the angular lifting member 210 may be a separate unit which is removably coupleable to both the attachment frame 212 and the medical device 300, such that the user can decide, while securing the attachment frame 212 to the patient's body, if the angular lifting member 210 is required or not.

In procedures involving insertion of a medical tool into the patient's body, in which use of an angular lifting member is required, an angular aiming apparatus may be used in order to facilitate proper positioning of the attachment frame which will ensure alignment of the medical tool tip with the marked entry point. The angular aiming apparatus may be coupled to the attachment frame and once proper positioning is achieved, the user can remove the angular aiming apparatus and couple the insertion device to the attachment frame. In some embodiments, the angular aiming apparatus may be provided with an orienting mechanism, for example similar to the orienting mechanism described with regard to FIGS. 8A and 8B hereinabove.

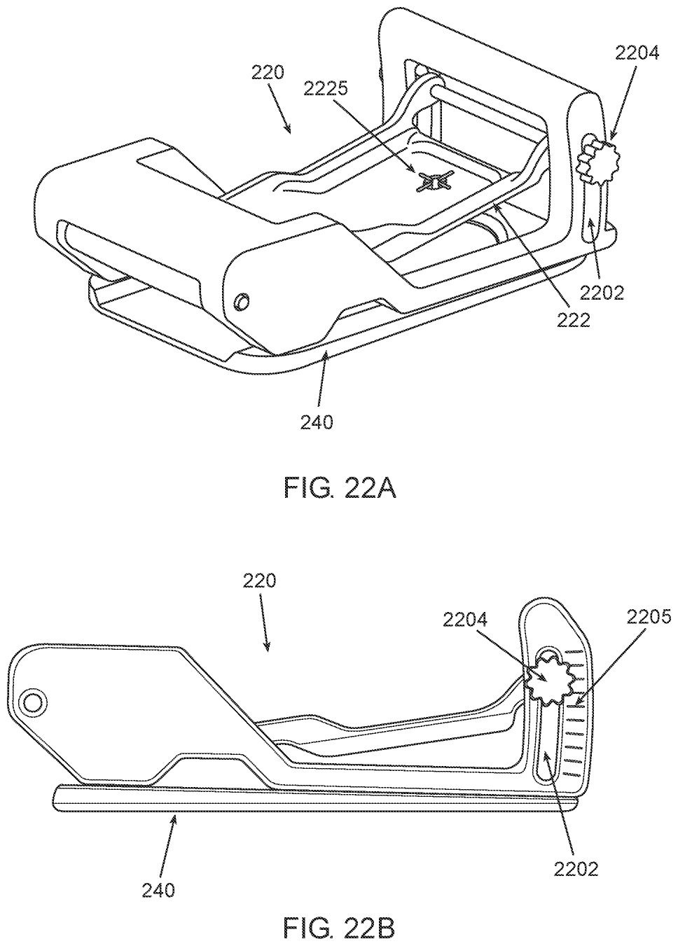

FIGS. 22A and 22B show an angular aiming apparatus 220 which has a base 222 having approximately the same dimensions as the attachment frame and as the base of the medical device. The angular aiming apparatus 220 may further include an opening 2225 in the base 222, which is located at a location corresponding to the expected location of the tip of the medical tool relative to the attachment frame 240 when the insertion device is coupled to the attachment frame 240. The opening 2225 may be provided with a cross, or any other mark, to further point to the expected location of the medical tool tip. In some embodiments, at least one of the proximal end and the distal end of the angular aiming apparatus 220 may comprise a vertical or arched rail 2202 along which the base 222 of the angular aiming apparatus 220 can be moved, and a fixator 2204, to fixate the position of the base 222 along the rail 2202, once the desired angle of the base 222 relative to the attachment frame 240, or to the body surface, has been reached. In some embodiments, markings/notches 2205 may be provided adjacent the rail 2202, to indicate to the user, and assist him/her in controlling, the rotation degree of the base 222.

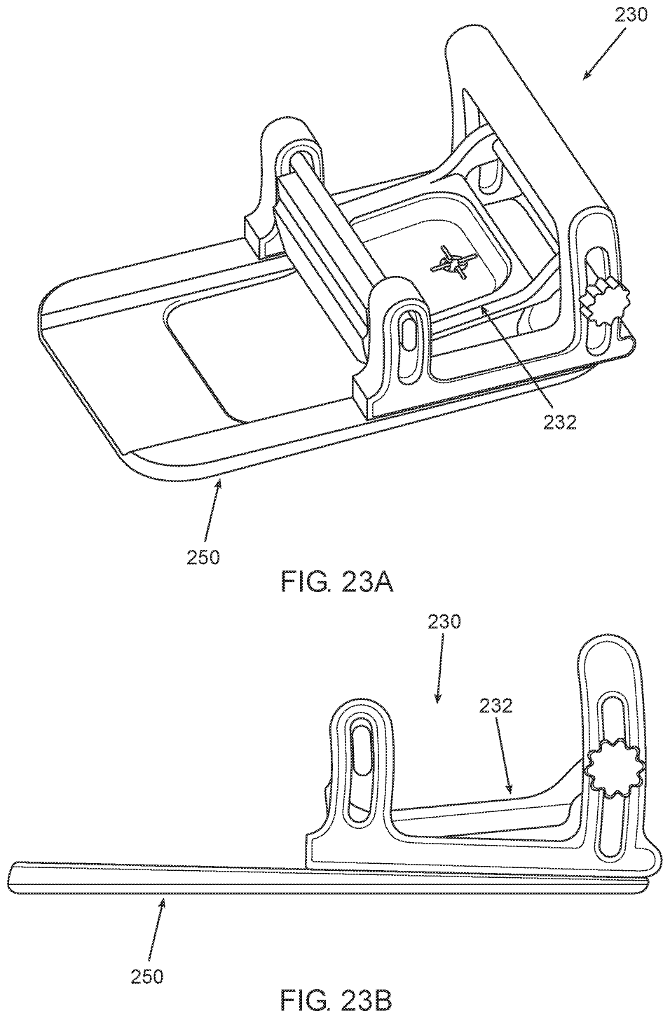

FIGS. 23A and 23B show an angular aiming apparatus 230 which has a base 232 which is smaller than the attachment frame 250 and the base of the medical device, and which is coupled to the attachment frame 250 at the end corresponding to the end where the medical tool will be located once the medical device is coupled to the attachment frame 250.

Although particular implementations have been disclosed herein in detail, this has been done by way of example for purposes of illustration only, and is not intended to be limiting with respect to the scope of the appended claims, which follow. In particular, it is contemplated that various substitutions, alterations, and modifications may be made without departing from the spirit and scope of the disclosure as defined by the claims. Other aspects, advantages, and modifications are considered to be within the scope of the following claims. The claims presented are representative of the implementations and features disclosed herein. Other unclaimed implementations and features are also contemplated. Accordingly, other implementations are within the scope of the following claims.

1.-31. (canceled)

32. An attachment apparatus for attaching a medical device to a body of a subject, the apparatus comprising:
an attachment frame configured for positioning on the body of the subject and for receiving the medical device thereon;
one or more extending members couplable to the attachment frame and configured to be extended outwardly from the attachment frame and retracted inwardly thereto;
one or more straps; and
one or more strap connectors configured to couple the one or more straps to at least one of the attachment frame and the one or more extending members.

33. The attachment apparatus of claim 32, wherein the attachment frame comprises at least one channel, and wherein at least one of the one or more extending members is reversibly deployable from within the at least one channel.

34. The attachment apparatus of claim 33, wherein the attachment frame comprises at least one locking member configured to lock the position of the at least one of the one or more extending members within the at least one channel.

35. The attachment apparatus of claim 32, wherein at least one of the one or more strap connectors is configured to enable pivoting of at least one of the one or more straps coupled thereto relative to the at least one of the attachment frame and the one or more extending members.

36. The attachment apparatus of claim 32, wherein at least one of the one or more strap connectors either comprises or is coupled to a stretching mechanism.

37. The attachment apparatus of claim 36, wherein the at least one of the one or more strap connectors comprises a first portion configured to be coupled to the at least one of the attachment frame and the one or more extending members, and a second portion configured to be coupled to at least one of the one or more straps and moveable relative to the first portion along a longitudinal axis of the at least one of the one or more strap connectors, and wherein the stretching mechanism comprises at least one elastic member coupled to both of the first and second portions.

38. The attachment apparatus of claim 36, wherein the at least one of the one or more strap connectors further comprises an indicator configured to indicate a stretching degree of the stretching mechanism.

39. The attachment apparatus of claim 32, wherein at least one of the one or more strap connectors comprises a locking mechanism configured to lock the position of at least one of the one or more straps relative to the at least one of the one or more strap connectors.

40. The attachment apparatus of claim 32, wherein the at least one of the attachment frame and the one or more extending members comprises one or more couplers configured to engage with the one or more strap connectors.

41. The attachment apparatus of claim 40, wherein the one or more strap connectors comprise ball connectors, and the one or more couplers comprise socket connectors configured to receive the ball connectors.

42. The attachment apparatus of claim 40, wherein the attachment frame comprises two frame couplers positioned at a first end of the attachment frame and configured to engage with the one or more strap connectors, and wherein two extending members are coupled to the attachment frame at a second end of the attachment frame, each of the two extending members comprising an extending member coupler configured to engage with the one or more strap connectors.

43. The attachment apparatus of claim 32, further comprising a harness configured for positioning between the body of the subject and a surface configured for the subject to lie thereon, and one or more harness connectors configured to couple the one or more straps to the harness.

44. The attachment apparatus of claim 32, wherein the attachment frame comprises a first portion of a coupling mechanism configured to couple the medical device to the attachment frame, and the medical device comprises a second portion of the coupling mechanism.

45. The attachment apparatus of claim 32, wherein the medical device is an insertion device comprising a medical tool configured for insertion into the body of the subject, and wherein the attachment apparatus further comprises an aiming apparatus removably couplable to the attachment frame, the aiming apparatus being configured to facilitate proper positioning of the attachment frame relative to an entry point for entry of the medical tool into the body of the subject.

46. A method for attaching a medical device to a body of a subject, comprising:
providing an attachment apparatus comprising:
an attachment frame configured for positioning on the body of the subject and for receiving the medical device thereon;
one or more extending members couplable to the attachment frame and configured to be extended outwardly from the attachment frame and retracted inwardly thereto;
one or more straps; and
one or more strap connectors configured to couple the one or more straps to at least one of the attachment frame and the one or more extending members;
positioning the attachment frame on the body of the subject;
using the one or more strap connectors, coupling the one or more straps to the at least one of the attachment frame and the one or more extending members; and
coupling the medical device to the attachment frame.

47. The method of claim 46, further comprising deploying at least one of the one or more extending members from within at least one channel of the attachment frame.

48. An attachment apparatus for attaching a medical device to a body of a subject, the apparatus comprising:
a back base configured for positioning between the body of the subject and a surface configured for the subject to lie thereon;
an attachment frame configured for positioning on the body of the subject and for receiving the medical device thereon, the attachment frame comprising at least one channel;
one or more extending members, at least one of the one or more extending members being configured for reversible deployment from within the at least one channel;
one or more straps configured to be connected to the back base and to at least one of the attachment frame and the one or more extending members;
one or more first connectors configured to connect a first end of the one or more straps to the at least one of the attachment frame and the one or more extending members; and
one or more second connectors configured to connect a second end of the one or more straps to the back base.

49. The attachment apparatus of claim 48, wherein at least one of the one or more first connectors is configured to enable pivoting of at least one of the one or more straps coupled thereto relative to the at least one of the attachment frame and the one or more extending members.

50. The attachment apparatus of claim 48, wherein the one or more first connectors comprise one or more ball connectors, and wherein the at least one of the attachment frame and the one or more extending members comprises one or more socket connectors configured to engage with the one or more ball connectors.

51. The attachment apparatus of claim 48, wherein at least one of the one or more first connectors either comprises or is coupled to a stretching mechanism.
